(19) United States
(12) Patent Application Publication
(10) Pub. No.: US 2026/0132265 A1
(43) Pub. Date: May 14, 2026

(54) PROCESSES FOR CONVERTING WASTE PLASTICS AND WASTE PLASTIC CONVERSION PLANTS

(71) Applicant: Chevron Phillips Chemical Company LP, The Woodlands, TX (US)

(72) Inventors: Joel MUTCHLER, Porter, TX (US); Richard HERNANDEZ, Spring, TX (US)

(21) Appl. No.: 18/947,132

(22) Filed: Nov. 14, 2024

(51) Int. Cl. C08J 11/16; B01J 8/02; B01J 8/20; B09B 3/40; B09B 3/70; B09B 3/80; B09B 101/75; C07C 4/06; C07C 4/10; C07C 4/22

(52) U.S. Cl. CPC C08J 11/16; B01J 8/0278; B01J 8/20; B09B 3/40; B09B 3/70; B09B 3/80; C07C 4/06; C07C 4/10; C07C 4/22; B09B 2101/75

(57) ABSTRACT

Aspects of the present disclosure generally relate to new processes for converting waste plastic. Aspects of the present disclosure also generally relate to new waste plastic conversion plants. In an aspect, a process for converting waste plastic is provided. The process includes heating a waste plastic feed with a dissolution medium to form a mixture comprising an insoluble component and a soluble component. The process further includes separating the soluble component of the mixture from the insoluble component of the mixture. The process further includes contacting the soluble component with a depolymerization catalyst to form a depolymerization product effluent. The process further includes separating a C12+ hydrocarbon from the depolymerization product effluent. The process further includes introducing the C12+ hydrocarbon to the dissolution unit.

### FIELD

Aspects of the present disclosure generally relate to new processes for converting waste plastic and to new waste plastic conversion plants.

### BACKGROUND

Waste plastic materials negatively impact the environment when disposed of in landfills after single use. From an environmental perspective, the need to recycle waste plastics is pressing. However, there is a lack of effective recycling options. A substantial challenge with conventional technologies for waste plastic recycling is the presence of different types of plastic and additives in the waste plastic. For example, the presence of halogen-and oxygen-containing polymers and additives in waste plastic leads to undesired side reactions. Here, these side reactions include the evolution of hydrochloric acid or other gases, as well as the formation of aldehydes and ketones. Not only does hydrochloric acid, aldehydes, and ketones hinder depolymerization of the waste plastic, such compounds contaminate the recycled end products due to formation of undesired cross-linking and undesired chain scission products. As an attempt to avoid this, conventional technologies expend high costs and energy to purify the waste plastic feed. In addition, upon melting mixed plastic waste that include different types of plastics, macrophase separation is observed, resulting in structural weaknesses and compromised materials properties in the recycled product.

Pyrolysis represents a traditional approach for waste plastic recycling. Here, waste plastic is converted to a pyrolysis oil useful for petrochemical processing as a feedstock or a fuel. However, the produced pyrolysis oil includes very high proportions of heavy wax materials that require additional processing prior to use as a feedstock or fuel. Cokers are also conventionally used for waste plastic recycling. Cokers thermally crack hydrocarbon molecules present in dissolved waste plastic. However, the excessive temperatures utilized with cokers and the fouling due to residual coke leads to excessive costs and undesired downtime for maintenance. Another conventional approach to waste plastic recycling includes fluid catalytic cracking (FCC) of a dissolved waste plastic to make gasoline components, propylene, and propane. The propylene and propane from an FCC unit are sent to olefin plants for olefin production or separated in fractionators to produce a C3 olefin feedstock. Besides the aforementioned drawbacks and other disadvantages present with these conventional approaches, none of these approaches are utilized for making, e.g., olefins generally or for making C2-C4 and naphtha feedstocks for olefin production.

There is a need for new processes for converting waste plastic. There is also a need for new waste plastic conversion plants.

### SUMMARY

Aspects described herein generally relate to new processes for converting waste plastic and to waste plastic conversion plants. The inventors found processes for the conversion of waste plastic to various output streams including C2-C4 hydrocarbons, C5-C11 hydrocarbons, and C12+ hydrocarbons. Advantageously, the C2-C4 hydrocarbons and C5-C11 hydrocarbons may be used as feedstocks for producing olefins and the C12+ hydrocarbons produced may be re-used and introduced into the process to aid in the dissolution of the waste plastic. Further, and in contrast to conventional technologies, aspects described herein eliminate the need to purify waste plastic prior to depolymerization. Additionally, relative to output streams produced by state-of-the-art technologies that are characterized as having contamination and poor materials' properties, aspects described herein may be utilized to limit side reactions, to produce hydrocarbon output streams having improved properties, or both.

In an aspect, a process for converting waste plastic is provided. The process includes heating, in a dissolution unit, a waste plastic feed with a dissolution medium to form a mixture comprising an insoluble component and a soluble component. The process further includes separating the soluble component of the mixture from the insoluble component of the mixture. The process further includes contacting the soluble component with a depolymerization catalyst under depolymerization conditions to form a depolymerization product effluent. The process further includes separating a C12+ hydrocarbon from the depolymerization product effluent, and introducing the C12+ hydrocarbon to the dissolution unit to heat with the waste plastic feed.

In another aspect, a process for converting waste plastic to hydrocarbons is provided. The process includes heating, under dissolution conditions and in a dissolution unit, a waste plastic feed with a dissolution medium to form a dissolution product, the dissolution conditions comprising a temperature in a range from about 100° C. to about 200° C. The process further includes performing a solid-liquid separation on the dissolution product to separate a soluble component of the dissolution product from an insoluble component of the dissolution product. The process further includes contacting, under depolymerization conditions, hydrocarbon polymers present in the soluble component with a depolymerization catalyst to form a depolymerization product effluent. The process further includes separating the depolymerization product effluent into at least three fractions comprising: a first fraction comprising a C2-C4 hydrocarbon; a second fraction comprising a C5-11 hydrocarbon; and a third fraction comprising a C12+ hydrocarbon. The process further includes combining the third fraction with the waste plastic feed in the dissolution unit.

In another aspect, a plant for converting waste plastic is provided. The plant includes a dissolution unit configured to heat a feed comprising waste plastic to provide a mixture comprising an insoluble component and a soluble component. The plant further includes a first separation unit configured to receive the mixture from the dissolution unit and to separate the mixture. The plant further includes a depolymerization reactor configured to receive the soluble component from the first separation unit and to contact the soluble component with a depolymerization catalyst under depolymerization conditions to form a depolymerization product effluent. The plant further includes a second separation unit configured to receive the depolymerization product effluent from the depolymerization reactor and to separate the depolymerization product effluent into a plurality of output streams. The plant further includes a line configured to transfer a first output stream of the plurality of output streams to the dissolution unit, the first output stream comprising a C12+ hydrocarbon.

### BRIEF DESCRIPTION OF THE DRAWINGS

So that the manner in which the above recited features of the present disclosure may be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, may admit to other equally effective aspects.

FIG. 1 is a flow diagram showing selected operations of a process for converting waste plastic according to at least one aspect of the present disclosure.

FIG. 2 is a generalized schematic flow diagram showing various implementations of processes described herein corresponding to operational areas or units in a waste plastic conversion plant according to at least one aspect of the present disclosure.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

Aspects of the present disclosure generally relate to new processes for converting waste plastic and to waste plastic conversion plants. The inventors found novel processing schemes that convert waste plastic into various liquid and gaseous hydrocarbon streams that may be upgraded to, for example, olefin feedstocks. The processing scheme may include heating a waste plastic feed to form a mixture, separating an insoluble component from the mixture, depolymerizing hydrocarbons present in a soluble component of the mixture, and separating the depolymerized hydrocarbons into a C2-C4 hydrocarbon stream, a C5-C11 hydrocarbon stream, and a C12+ hydrocarbon stream. The C12+ hydrocarbon stream may be reintroduced to the process to dissolve waste plastic feeds that are converted using processes described herein. The C2-C4 hydrocarbon stream and the C5-C11 hydrocarbon stream may be utilized as feedstocks for producing olefins. The processes enable, for example, economically efficient conversion of waste plastics.

As described herein, conventional technologies for depolymerizing waste plastic have significant issues with impurities and side reactions as a result of mixed polymer types present in waste plastic feeds. For example, contaminants such as fillers and other additives or undesirable polymers such as polyvinylchloride (PVC) present in a polyolefin waste plastic feed may adversely affect the depolymerization process. Here, depolymerization of PVC leads to release of hydrochloric acid (HCl) which may undesirably combine with depolymerized polyolefins and contaminate entire batches of polymers. Conventional attempts to avoid these unwanted side products include substantial purification prior to depolymerization. Even so, conventional technologies include units downstream of depolymerization for reducing the concentration of, or removing, halogenated components.

Advantageously, aspects described herein may be used to eliminate the need to purify waste plastic feeds prior to depolymerization and may be utilized to prevent, or at least mitigate, undesired side reactions that abate end materials' properties. For example, and as described herein, waste plastic feeds may be selectively dissolved under selected conditions and then subjected to removal of insoluble components. By controlling the temperature during heating of the waste plastic feed and/or separating insoluble components prior to depolymerization, the undesired reactions may be minimized and the end materials' properties may be improved relative to conventional waste plastic conversion technologies.

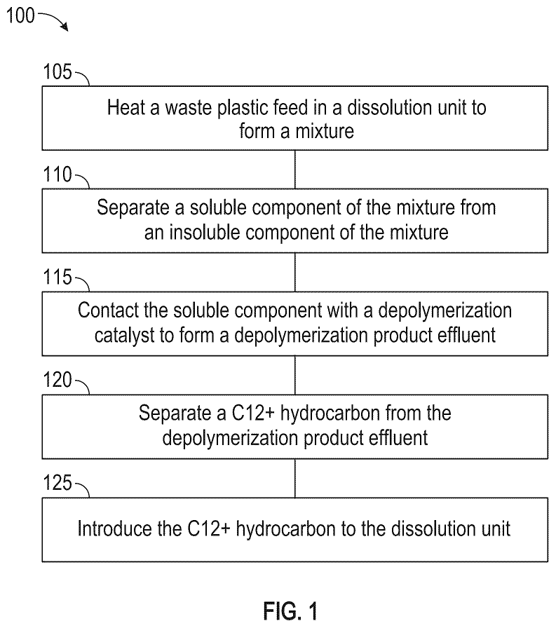

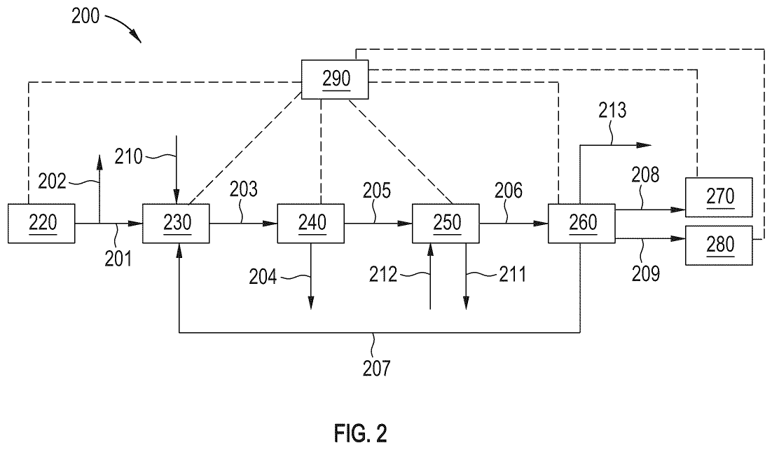

Aspects of the present disclosure generally relate to new processes for converting waste plastic. FIG. 1 is a flow diagram showing selected operations of a process 100 for converting waste plastic according to at least one aspect. Process 100 may be utilized to convert any suitable waste plastic feed. Aspects and implementations of process 100 may be combined with other aspects and implementations described herein, such as aspects and implementations described with respect to FIG. 2. In general, processes of the present disclosure enable conversion of waste plastic to various hydrocarbons or hydrocarbon streams that may be utilized for any suitable purpose such as for producing olefins. The term “olefin” is used herein in accordance with the definition specified by IUPAC: acyclic and cyclic hydrocarbons having one or more carbon-carbon double bonds apart from the formal ones in aromatic compounds. The class “olefins” subsumes alkenes and cycloalkenes and the corresponding polyenes. Ethylene, propylene, 1-butene, 2-butene, 1-hexene, and the like are non-limiting examples of olefins.

The process 100 may begin with heating a waste plastic feed in a dissolution unit to form a mixture that includes an insoluble component and a soluble component at operation 105. The mixture resulting from operation 105 is interchangeably referred to as a dissolution product.

The waste plastic feed may generally include polymers such as olefin polymers (polyolefins). Suitable polyolefins may include polyolefins made from an alpha olefin. The term “alpha olefin” as used herein refers to an olefin that has a double bond between the first and second carbon atom of the longest contiguous chain of carbon atoms. The term “alpha olefin” includes linear and branched alpha olefins unless expressly stated otherwise. Suitable polyolefins present in the waste plastic feed may include polyethylene, polypropylene, polystyrene, other polyolefins, or combinations thereof. The polyolefins present in the waste plastic feed may include a polyolefin homopolymer, a polyolefin copolymer, a polyolefin block copolymer, a polyolefin random block copolymer, or combinations thereof. The polyolefins present in the waste plastic feed may include an isotactic polyolefin, an atactic polyolefin, a syndiotactic polyolefin, or combinations thereof. Suitable polyethylenes present in the waste plastic feed may include high density polyethylene, medium density polyethylene, low density polyethylene, linear low density polyethylene, or combinations thereof. Polyolefin copolymers and terpolymers are contemplated. As used herein, a copolymer is derived from an olefin monomer and one olefin comonomer, while a terpolymer is derived from an olefin monomer and two olefin comonomers. As an example of polyolefin copolymers, an ethylene copolymer may be derived from ethylene and a comonomer, such as 1-butene, 1-hexene, or 1-octene. Suitable polypropylenes present in the waste plastic feed may include polypropylene homopolymer, polypropylene random block copolymer, or combinations thereof.

An amount of polyolefin present in the waste plastic feed may be about 10 wt % or more, such as in a range from about 10 wt % to about 99 wt %, such as from about 20 wt % to about 98 wt %, such as from about 40 wt % to about 95 wt %, such as from about 50 wt % to about 90 wt %, such as from about 70 wt % to about 85 wt % based on a total weight of the waste plastic feed. The total weight of the waste plastic feed is 100 wt %.

The waste plastic feed may include a polyolefin waste without additional waste plastics or other components or compositions being present in the waste plastic feed. The waste plastic feed may include polyolefin and other types of waste plastic which are different from the polyolefin. For example, in addition to the polyolefin, the waste plastic feed may include an oxygen-containing polymer, a halogen-containing polymer, or combinations thereof. Illustrative, but non-limiting, examples of oxygen-containing polymers that may be present in the waste plastic feed may include: a polyester such as polyethylene terephthalate, polybutylene terephthalate, or a combination thereof; a polyamide; a polyurethane; a polyphenol; a polycarbonate; a polylactic acid; a polyacrylic acid; a polyacrylate; a polyacetal; a halogen-containing polymer; or combinations thereof. As used herein, and unless specified to the contrary or the context clearly indicates otherwise, the term “halogen” refers to fluorine, chlorine, bromine, and iodine, regardless of whether these elements are in neutral or anionic form or occur as molecular or polymeric substituents or atoms in a solid-state structure. Illustrative, but non-limiting, examples of halogen-containing polymers that may be present in the waste plastic feed may include: a chlorinated polymer such as chlorinated polyethylene; polyvinylchloride (PVC), polyvinylidene chloride (PVDC); a fluorinated polymer such as fluorinated polyethylene, such as polytetrafluoroethylene; or combinations thereof.

When the waste plastic feed includes waste plastic other than the polyolefin, an amount of the waste plastic other than the polyolefin may be about 10 wt % or less, about 0.5 wt % or more, or a combination thereof, such as in a range from about 1 wt % to about 10 wt %, such as from about 2 wt % to about 5 wt % based on a total weight of the waste plastic feed.

PET and PVC are prevalent in waste plastics. In various aspects, which may be combined with other aspects, the waste plastic feed may include a polyolefin, and optionally one or more additional waste plastics other than polyolefin such as PVC, PET, or a combination thereof.

The polyester, if present in the waste plastic feed, may include polyethylene terephthalate (PET), polybutylene terephthalate, or a combination thereof, among other polyesters. When the waste plastic feed includes a polyester, the polyester may be present in the waste plastic feed in an amount of about 10 wt % or less, such as about 5 wt % or less, such as about 2 wt % or less, such as about 1 wt % or less, such as about 0.5 wt % or less based on the total weight of the waste plastic feed.

When the waste plastic feed includes PVC, the PVC may be present in the waste plastic feed in an amount of about 10 wt % or less, such as about 5 wt % or less, such as about 2 wt % or less, such as about 1 wt % or less, such as about 0.5 wt % or less, or in a range from about 2 wt % to about 7.5 wt %, such as from about 3.5 wt % to about 6 wt % based on the total weight of the waste plastic feed. Many times, mixed waste plastic may include a PVC content of about 3.5 wt % to about 6 wt % and chloride content is about 60% by weight.

The waste plastic feed may be characterized as having a certain halogen content. The waste plastic feed may include a halogen content, if present, in an amount of about 36,000 ppm (about 3.6 wt %) or less, such as about 10,000 ppm (about 1 wt %) or less, or in a range from greater than 0 ppm to about 36,000 ppm, such as from greater than 0 ppm to about 10,000 ppm based on a total weight of the waste plastic feed.

The waste plastic feed may further include a virgin plastic. Virgin plastic refers to a new unused plastic material and is not manufactured from reprocessed materials. Virgin plastics have not been previously blended with scrap, waste, or previously used material. The virgin plastic may serve to aid in processing the waste plastic feed. When the waste plastic feed includes virgin plastic, the virgin plastic may be present in the waste plastic feed in an amount of about 10 wt % or less, such as about 5 wt % or less, such as about 2 wt % or less, such as about 1 wt % or less, such as about 0.5 wt % or less based on the total weight of the waste plastic feed.

The waste plastic feed may be solid at 25° C. The waste plastic feed may include a solid characterized by an average particle size of about 10 centimeters (cm) or less, such as about 1 cm or less, such as about 100 millimeters (mm) or less, such as about 10 mm or less, such as about 5 mm or less, such as about 3 mm or less, such as about 1 mm or less, such as about 500 microns (μm) or less, such as about 10 μm or less. The waste plastic feed may be in any suitable form such as a pellet, a flake, a fluff, a particle, or combinations thereof. The waste plastic feed may include a solid in pellet form. The solid in pellet form may be characterized by an average particle size in a range from about 0.01 mm to about 10 mm, such as from about 0.1 mm to about 1 mm. The waste plastic feed may include a solid in fluff form. The solid in fluff form may be characterized by an average particle size of about 1,500 microns (μm) or less, such as about 1,000 μm or less, such as about 500 μm or less, such as about 400 μm or less, such as about 300 μm or less, such as about 100 μm or less, such as about 50 μm or less, such as about 10 μm or less.

Typically, plastics include additives that provide plastics with various properties for desired end uses. Accordingly, the waste plastic feed may further include various additives used during the manufacture of plastics, for example: a rubber such as nitrile rubber; a filler such as calcium carbonate, talk, carbon black, or glass fiber; a pigment such as titanium dioxide; a dye, a process aid, a stabilizer, an ink, a plasticizer, a slip agent, an antiblock agent, an antioxidant, a ultraviolet (UV) light stabilizer, a cellulose fiber, an adhesive, or combinations thereof.

Referring back to operation 105, the waste plastic feed may be heated in the dissolution unit in the presence of a dissolution medium. The dissolution medium may include a hydrocarbon feed. The hydrocarbon feed may be a hydrocarbon mixture having a boiling point in a range from about 214° C. to about 680° C., such as from about 250° C. to about 600° C., such as from about 300° C. to about 500° C., such as from about 350° C. to about 450° C. Additionally, or alternatively, the hydrocarbon feed may include, for example: a petroleum-based material such as vacuum gas oil (VGO); a fossil fuel-based material; a bio-based material; or combinations thereof. Additionally, or alternatively, and as described herein, the waste plastic feed may be heated in the presence of a C12+ hydrocarbon produced by implementations of the process 100. The C12+ hydrocarbon produced by implementations of the process 100 may include a C12+ paraffin, a C12+ olefin, a C12+ aromatic, or combinations thereof.

The heating process of operation 105 may include heating the waste plastic feed, optionally in the presence of the dissolution medium and/or the C12+ hydrocarbon, under dissolution conditions effective to dissolve, or selectively dissolve, at least a portion of the waste plastic feed. Such dissolution conditions effective to dissolve, or selectively dissolve, at least a portion of the waste plastic feed may include heating at a dissolution temperature that is about 250° C. or less, such as in a range from about 75° C. to about 225° C., such as from about 110° C. to about 200° C., such as from about 130° C. to about 180° C., such as from about 140° C. to about 160° C., or in a range from about 100° C. to about 130° C., such as from about 100° C. to about 110° C. In some aspects, which may be combined with other aspects, the waste plastic feed may be heated at a dissolution temperature that is below the boiling point of the dissolution medium, such as at a temperature that is from about 8° C. to about 50° C. below the boiling temperature of the dissolution medium. In some aspects, which may be combined with other aspects, the waste plastic feed may be heated at a dissolution temperature that is below the boiling point of the C12+ hydrocarbon produced by process 100, such as a temperature that is from about 8° C. to about 50° C. below the boiling temperature of the C12+ hydrocarbon produced by process 100. Optionally, dissolution conditions to dissolve, or selectively dissolve, at least a portion of the waste plastic may include a dissolution pressure in a range from about 100 kiloPascals (kPa) to about 2,000 kPa, such as from about 500 kPa to about 1,500 kPa, such as from about 750 kPa to about 1,250 kPa, or in a range from about 345 kPa to about 2,068 kPa, such as from about 1,724 kPa to about 2,068 kPa.

The dissolution unit utilized in operation 105 may include any suitable reactor or vessel such as a continuous stirred tank reactor. The dissolution conditions to dissolve, or selectively dissolve, at least a portion of the waste plastic feed may include any suitable residence time that the waste plastic feed is present in the dissolution unit. For example, the waste plastic feed may be present in the dissolution unit for a residence time that may be in a range from about 20 minutes to about 60 minutes, such as from about 25 minutes to about 35 minutes. The dissolution conditions of operation 105 may further include operating the reactor or vessel at any suitable mixing speed such as in a range from about 100 revolutions per minute (rpm) to about 1,000 rpm, such as from about 500 rpm to about 1,000 rpm.

One or more of the aforementioned dissolution conditions may be utilized to minimize dissolution of waste plastic other than the polyolefin, to minimize dissolution of one or more additives used during manufacture of plastics, or a combination thereof. For example, the dissolution conditions may minimize dissolution of a halogen-containing compound such as a halogen-containing polymer, a halogen-containing additive, or combinations thereof present in the waste plastic feed, such as those halogen-containing polymers described herein as well as various halogen-containing additives used in the production of plastics. Additionally, or alternatively, the aforementioned dissolution conditions may be utilized to minimize dissolution of an oxygenated compound such as an oxygen-containing polymer, to minimize dissolution of an oxygen-containing additive, or combinations thereof, such as those oxygen-containing polymers described herein as well as various oxygen-containing additives used in the production of plastics. The one or more dissolution conditions may also be utilized to control or limit side reactions, such as those side reactions described herein, that are detrimental to downstream depolymerization and end materials' properties.

Prior to operation 105 of process 100, the waste plastic feed may be optionally mechanically treated. Mechanical treatment may include comminution. In general, comminution allows the waste plastic to be broken into pieces of various sizes by, for example, crushing, grinding, cutting, vibrating, shredding, pelletizing, granulating, hammering, other comminution processes, or combinations thereof. The waste plastic feed may be comminuted with a crusher, a grinder, a cutter, a vibrator, a shredder, a pelletizer, a granulator, a hammer mill, or combinations thereof. The waste plastic feed may be comminuted into, for example, a pellet, a flake, a fluff, a particle, or combinations thereof.

Waste plastic feed may include water, O₂, or combinations thereof. Accordingly, and prior to heating the waste plastic feed at operation 105, the process may optionally include removing at least a portion of water from the waste plastic feed, removing at least a portion of oxygen (O₂) from the waste plastic feed, or a combination thereof. Water may be removed from the waste plastic feed by any suitable technique such as by thermal drying, hydraulically pressing, centrifugal dewatering, or combinations thereof. O₂ may be removed from the waste plastic feed by any suitable technique such as by purging the waste plastic feed with a non-reactive gas such as N₂, Ar, or a combination thereof. Following removal of at least a portion of the water from the waste plastic feed, the waste plastic feed may include about 5,000 ppm (0.5 wt %) or less of water, such as in a range from greater than 0 ppm to about 5,000 ppm based on the total weight of the plastic feed. Following removal of at least a portion of the O₂ from the waste plastic feed, the waste plastic feed may include about 5,000 ppm (0.5 wt %) or less of O₂, such as in a range from greater than 0 ppm to about 5,000 ppm based on the total weight of the plastic feed. The optional removal of water, O2, or a combination thereof may occur before, after, or concurrently with the optional mechanical treatment.

Referring back to FIG. 1, the process 100 may further include separating the soluble component of the mixture from the insoluble component of the mixture at operation 110. Soluble components present in the mixture may include C1-C50 hydrocarbon polymers, such as C1-C30 hydrocarbon polymers.

Insoluble components, such as solids, which may be present in the mixture may include waste plastic other than polyolefin. Waste plastics other than the polyolefin, and which may be present in the insoluble component, are described herein, and may include a halogen-containing polymer, an oxygen-containing polymer, or a combination thereof. Additionally, or alternatively, insoluble components which may be present in the mixture may include additives used in the manufacture of plastics, for example, a rubber, a filler, a pigment, a dye, a process aid, a stabilizer, an ink, a plasticizer, a slip agent, an antiblock agent, an antioxidant, a UV stabilizer, a cellulose fiber, an adhesive, or combinations thereof, such as halogen-containing additives, oxygen-containing additives, or combinations thereof. Additionally, or alternatively, insoluble components which may be present in the mixture may include heavy hydrocarbons, such as C30+ hydrocarbons, such as C30-C50 hydrocarbons. The C30-C50 hydrocarbons may be waxes.

The separation process of operation 110 may be performed using any suitable technique such as solid-liquid separation techniques. Illustrative, but non-limiting, solid-liquid separation techniques useful for operation 110 may include filtration, vacuum filtration, centrifugation, decantation, decanting centrifugation, or combinations thereof, among other techniques. The separation process may serve to remove at least a portion of those insoluble components described above. For example, the separation process of operation 110 may be utilized to remove at least a portion of additives, C30+ hydrocarbons, waste plastics other than polyolefin, or combinations thereof, if present, in the waste plastic feed. The separation process of operation 110 may be utilized to remove at least a portion of halogenated compounds—such as halogen-containing polymers described herein, halogen-containing additives, or a combination thereof (if present)—in the waste plastic feed. The separation process of operation 110 may be utilized to remove at least a portion of oxygenated compounds—such as oxygen-containing polymers described herein, oxygen-containing additives, or a combination thereof (if present)—in the waste plastic feed.

By, for example, selectively dissolving polyolefins present in the waste plastic feed at selected temperatures and removing insoluble component(s) from the mixture, downstream depolymerization is more effective and the end products from process 100 are more pure and useful due to the prevention, or at least mitigation, of undesired side reactions. Such undesired side reactions present with conventional processes include formation of HCl or other gases that evolve during heating. Aldehydes and ketones are also formed during conventional conversion of waste plastic. These undesired compounds may hinder depolymerization and may contaminate the end products of process 100. For example, HCl, aldehydes, and ketones, can initiate cross-linking and chain-scission reactions that abate materials properties.

The process 100 may further include contacting the soluble component with a depolymerization catalyst to form a depolymerization product effluent at operation 115. The soluble component utilized in the depolymerization includes hydrocarbon polymers such as C1-C50 hydrocarbon polymers. Through contact of the hydrocarbon polymers with the depolymerization catalyst, the polymer skeleton of the hydrocarbon polymers depolymerizes, degrades, or decomposes to smaller hydrocarbons by, for example, chain scission and/or additional reactions. Chain scission refers to breaking of covalent bonds that make up the molecular structure of the hydrocarbon polymer.

Any suitable depolymerization catalyst may be utilized at operation 115. Suitable depolymerization catalysts may include a zeolite-based catalyst, a chromia-based catalyst, a non-zeolite cracking catalyst, a solid acid catalyst, or combinations thereof. The depolymerization catalyst may achieve depolymerization of hydrocarbons present in the soluble component, but the depolymerization catalyst may also carry out additional reactions. For example, the depolymerization catalyst may achieve dehydrogenation, hydrogenolysis, cracking, isomerization, dehydrohalogenation, dehalogenation, or a combination thereof, of hydrocarbons present soluble component.

Suitable zeolite-based catalysts useful as depolymerization catalysts for operation 115 may include L-zeolite (zeolite L or LTL), X-zeolite (zeolite X), Y-zeolite (Zeolite Y), omega zeolite, beta zeolite, SAPO-34 zeolite, USY Zeolite, HY zeolite, ZSM-4, ZSM-5 (MFI), ZSM-10, ZSM-11, ZSM-12, ZSM-20, ZSM-22, ZSM-23, ZSM-34, ZSM-35, ZSM-50, REY, USY, RE-USY, LZ-210, LZ-210-A, LZ-210-M, LZ-210-T, SSZ-13, SSZ-24, SSZ-26, SSZ-31, SSZ-33, SSZ-35, SSZ-37, SSZ-41, SSZ-42, SSZ-44, MCM-58, H-MOR (H-mordenite), mazzite, faujasite, chabazite, a modified mesoporous form thereof, an acid-modified form thereof, or combinations thereof.

The zeolite-based catalyst may further include any suitable transition metal. The transition metal is utilized to effect the depolymerization. The transition metal of the zeolite-based catalyst may include chromium (Cr), molybdenum (Mo), tungsten (W), iron (Fe), ruthenium (Ru), osmium (Os), cobalt (Co), rhodium (Rh), iridium (Ir), nickel (Ni), palladium (Pd), platinum (Pt), gold (Au), zinc (Zn), titanium (Ti), tantalum (Ta), or combinations thereof, such as Ru, Os, Rh, Ir, Pd, Pt, Au, or combinations thereof.

The zeolite-based catalyst may further include a binder. Binders may be used to shape the zeolite-based catalyst and to provide mechanical strength and resistance to attrition loss. The type of binder utilized may have an impact on the catalytic performance of the zeolite-based catalyst. Any suitable binder may be utilized such as an inorganic oxide. Illustrative, but non-limiting, inorganic oxides useful as binders for the zeolite-based catalyst may include silica, alumina, silica-alumina, clay, titania, magnesium oxide, or combinations thereof. In some aspects, which may be combined with other aspects, the zeolite-based catalyst with a silica binder can be prepared from a silica sol. In some aspects, which may be combined with other aspects, a zeolite-based catalyst with an alumina binder may be prepared from a solution. The resulting pastes from these mixtures may be fired to make the bound zeolite-based catalysts. The zeolite-based catalyst may include one or more zeolites and any suitable amount of binder that may provide the zeolite-based catalyst in a suitable solid structure after processing. For example, the zeolite-based catalyst may include the zeolite or combinations of zeolites and from about 15 wt % to about 35 wt % binder, alternatively from about 20 wt % to about 30 wt % binder, or alternatively any weight percentage between these values.

Illustrative, but non-limiting, zeolite-based catalysts useful for depolymerization may include Pt-L-zeolite, Pt-ZSM-5, Pt-Y zeolite, Pt-SAPO-34 zeolite, Pt-USY zeolite, Pt-HY zeolite, Pt-beta zeolite, or combinations thereof.

The zeolite-based catalyst may further include a promoter. Promoters may help tailor the product distribution of the depolymerization product effluent and may be utilized to influence the activity of the zeolite-based catalyst. Any suitable promoter may be utilized such as those promoters that include a Group 1 metal, a Group 2 metal, a Group 14 metal, a Group 15 metal, a Group 15 non-metal, or combinations thereof, of the periodic table of the elements. For example, the promoter may include tin, bismuth, phosphorous, or combinations thereof. When the promoter includes tin, the tin content may be imparted by treating the zeolite or the zeolite-based catalyst itself with a tin salt or compound, such as stannous chloride, stannic chloride, stannic tartrate, stannic nitrate, or combinations thereof, according to suitable methods. When the promoter includes phosphorous, the zeolite or the zeolite-based catalyst itself may be treated with a phosphorous compound, such as phosphoric acid, according to suitable methods.

The zeolite of the zeolite-based catalyst may be characterized as having any suitable pore volume. For example, the zeolite of the zeolite-based catalyst may be characterized as having a zeolite pore volume in a range from about 0.10 milliliters per gram (mL/g) to about 2.0 mL/g, such as from about 0.25 mL/g to about 1.75 mL/g, such as from about 0.5 mL/g to about 1.5 mL/g, such as from about 0.75 mL/g to about 1.25 mL/g, such as about 1.0 mL/g. These quantitative pore volumes are features of the starting zeolite itself prior to forming the catalyst using the transition metals and promoters as described herein.

Zeolite pore diameters of the zeolite-based catalyst may vary and may be any suitable zeolite pore diameter. For example, the zeolite of the zeolite-based catalyst may be characterized as having a zeolite pore diameter in a range from about 3.0 Angstroms (Å) to about 10.0 Å, such as from about 4.0 Å to about 9.0 Å, such as from about 5.0 to about 8.0, such as from about 5.5 Å to about 7.0 Å. Additionally, or alternatively, the zeolite of the zeolite-based catalyst may be characterized as having a zeolite pore diameter in a range from about 7.0 Å to about 10.0 Å, or from about 5.0 Å to about 6.0 Å, or from about 2.0 Å to about 3.0 Å. These quantitative pore diameters are features of the starting zeolite itself prior to forming the catalyst using the transition metals and promoters as described herein.

The zeolite-based catalyst may be characterized as having any suitable catalyst surface area. For example, the zeolite-based catalyst may be characterized as having a catalyst surface area in a range from about 100 meters squared per gram (m²/g) to about 1,000 m²/g, such as from about 200 m²/g to about 900 m²/g, such as from about 300 m²/g to about 800 m²/g, such as from about 400 to about 700 m²/g, such as from about 500 m²/g to about 600 m²/g.

The zeolite-based catalyst may be in particulate form and may have an average particle size in a range from about 1 microns (μm) to about 100 μm, such as from about 2 μm to about 50 μm, such as from about 3 μm to about 30 μm, such as from about 4 μm to about 20 μm, or from about 5 μm to about 20 μm. For example, the zeolite-based catalyst may include a Y-zeolite and may be in particulate form having an average particle size in a range from about 3 μm to about 8 μm. As another example, the zeolite-based catalyst may include ZSM-5 (MFI) and may be in particulate form having an average particle size in a range from about 5 μm to about 10 μm. As another example, the zeolite-based catalyst may include H-MOR and may be in particulate form having an average particle size in a range from about 12 μm to about 18 μm. As another example, the zeolite-based catalyst may include beta zeolite and may be in particulate form having an average particle size in a range from about 3 μm to about 5 μm. One or more zeolite-based catalysts may be used as the depolymerization catalyst.

The depolymerization catalyst useful for operation 115 may include a chromia-based catalyst. Suitable chromia-based catalysts may include a chromium compound such as an organic chromium compound, a chromium oxide, or a combination thereof. An illustrative, but non-limiting, example of an organic chromium compound may include chromium acetylacetonate (CrAcAc). Suitable chromium oxides may include, for example, amorphous Cr₂O₃, crystalline Cr₂O₃, or a combination thereof. The chromia-based catalyst may be supported or may be free of a support. Suitable supports for the chromia-based catalyst may include silica, silica-alumina, silica-coated alumina, silica-titania, silica-magnesia, alumina, zirconia, thoria, mixed oxides thereof, or combinations thereof.

For example, the chromia-based catalyst may include: CrAcAc; chromia-alumina; chromia-magnesia-alumina; magnesium chromite-tin oxide; magnesium chromite-alumina-tin oxide; magnesium chromite combined with a promoter comprising one or more of boron (B), silicon (Si), tin (Sn), lead (Pb), Zn, or selenium (Se); or combinations thereof. The B, Si, Sn, Pb, Zn, or Se promoter may be present from 0.1 wt % to 10 wt % relative to the combined chromium compound (e.g., on a CrAcAc basis or Cr₂O₃ basis) and the promoter.

The depolymerization catalyst useful for operation 115 may include any suitable non-zeolite cracking catalyst. Suitable non-zeolite cracking catalysts may include clay, acid treated clay, perovskite, layered titanate, silica alumina, silica-coated alumina, acidic alumina, tungstated zirconia, activated carbon, natural kaolin, acid-modified kaolin, bentonite, or combinations thereof.

The depolymerization catalyst useful for operation 115 may include any suitable solid acid catalyst, such as a solid super acid (SSA). Such solid acid catalysts include a solid oxide treated with an electron withdrawing anion. The solid acid catalyst may be used in addition to another depolymerization catalyst such as a zeolite-based catalyst. For example, after separating the soluble component of the mixture from the insoluble component of the mixture and prior to the contacting the soluble component with a depolymerization catalyst comprising a zeolite-based catalyst (or other depolymerization catalyst), the process 100 may further include, contacting the soluble component with a solid acid catalyst. The solid acid catalyst may provide improvements in the depolymerization reaction of operation 115. The solid catalyst may provide substantial acidity such as Lewis acidity. While not wishing to be bound by any theory, it is believed that an SSA may start the process of degrading the polyolefins into shorter polymer or oligomer chains sooner, which may enhance the depolymerization reaction in the presence of the zeolite-based catalyst or other depolymerization catalyst.

Any suitable solid oxide treated with an electron withdrawing anion may be utilized as the solid acid catalyst. For example, the solid oxide of the solid oxide treated with the electron withdrawing anion may include silica, alumina, silica-alumina, silica-coated alumina, aluminum phosphate, aluminophosphate, heteropolytungstate, titania, magnesia, boria, zinc oxide, silica-zirconia, silica-titania, a mixed oxide thereof, or combinations thereof. The electron-withdrawing anion used for treating the solid oxide may include sulfate, bisulfate, fluorosulfate, phosphate, fluorophosphate, fluoride, chloride, bromide, or combinations thereof. The solid oxide treated with the electron withdrawing anion may include a sulfated solid oxide, a fluorided solid oxide, a phosphated solid oxide, a fluorophosphated solid oxide, a chlorided solid oxide, a bromided solid oxide, or a combinations thereof.

The solid oxide treated with the electron withdrawing anion may include fluorided alumina, chlorided alumina, bromided alumina, phosphated alumina, sulfated alumina, fluorided silica-alumina, chlorided silica-alumina, bromided silica-alumina, sulfated silica-alumina, fluorided silica-coated alumina, sulfated silica-coated alumina, phosphated silica-coated alumina, fluorophosphated silica-coated alumina, fluorided silica-titania, fluorided silica-zirconia, chlorided silica-zirconia, bromided silica-zirconia, sulfated silica-zirconia, fluorided silica-zirconia, or combinations thereof, such as fluorided alumina, sulfated alumina, fluorided silica-alumina, sulfated silica-alumina, fluorided silica-coated alumina, phosphated silica-coated alumina, sulfated silica-coated alumina, or combinations thereof.

Contact of the soluble component with the depolymerization catalyst at operation 115 may be performed under any suitable depolymerization conditions. The depolymerization conditions are effective to depolymerization at least a portion of hydrocarbons present in the soluble component. Depolymerization conditions of operation 115 may include heating at a depolymerization temperature in a range from about 200° C. to about 800° C., such as from about 300° C. to about 700° C., such as from about 400° C. to about 600° C., such as from about 450° C. to about 550° C., or in a range from about 200° C. to about 300° C., such as from about 200° C. to about 250° C., while contacting the soluble component with the depolymerization catalyst. Depolymerization conditions may include the use of hydrogen. For example, depolymerization conditions at operation 115 may include contacting the soluble component with the depolymerization catalyst in the presence of hydrogen at a pressure in a range from about 34 kPa to about 6,895 kPa, such as from about 335 kPa to about 5,516 kPa, such as from about 689 kPa to about 2,069 kPa, such as from about 1,034 kPa to about 1,724 kPa. However, the depolymerization conditions may include contacting the soluble component and the depolymerization catalyst at the depolymerization temperature in the absence of hydrogen. The depolymerization conditions at operation 115 may further include contacting the soluble component with the depolymerization catalyst for a duration sufficient to depolymerize at least a portion of hydrocarbons present in the soluble component.

Depolymerization may be performed in any suitable reactor. For example, contacting the soluble component with the depolymerization catalyst at operation 115 may be performed in a fixed bed reactor or a fluidized bed reactor. In a fixed bed reactor, depolymerization catalyst pellets are held in place and do not move with respect to a fixed reference frame with reactants flowing through the bed and being converted into products. In the fixed bed reactor, the depolymerization catalyst may have any suitable configuration including, but not limited to, one large bed, several horizontal beds, several parallel packed tubes, or multiple beds in their own shells. The various configurations may be adapted depending on, for example, a desire to maintain temperature control within the system. Fluidized bed reactors are catalytic reactors in which the mass of the catalyst is fluidized. This allows for extensive mixing in all directions. A result of the extensive mixing may include excellent temperature stability, uniform mixing, and increased mass transfer and reaction rates. A distinguishing feature of a fluidized bed reactor is that the bed of solid particles or catalyst is fluidized by an upflow of gas.

The depolymerization product effluent forms as a result of contact of the soluble component with the depolymerization catalyst at operation 115. The depolymerization effluent may include various hydrocarbons. For example, the depolymerization effluent may include a C2-C4 hydrocarbon, a C5-C11 hydrocarbon (for example, naphtha), a C12+ hydrocarbon, and optionally methane.

“A C2-C4 hydrocarbon” includes aspects comprising one, two, or more C2-C4 hydrocarbons, unless specified to the contrary or the context clearly indicates only one C2-C4 hydrocarbon is included. The C2-C4 hydrocarbon may include one or more C2-C4 paraffins, one or more C2-C4 olefins, or combinations thereof. “A C5-C11 hydrocarbon” includes aspects comprising one, two, or more C5-C11 hydrocarbons, unless specified to the contrary or the context clearly indicates only one C5-C11 hydrocarbon is included. The C5-C11 hydrocarbon may include one or more C5-C11 paraffins, one or more C5-C11 olefins, one or more C5-C11 aromatics, or combinations thereof. “A C12+ hydrocarbon” includes aspects comprising one, two, or more C12+ hydrocarbons, unless specified to the contrary or the context clearly indicates only one C12+ hydrocarbon is included. The C12+ hydrocarbon may include one or more C12+ paraffins, one or more C12+ olefins, one or more C12+ aromatics, or combinations thereof.

The depolymerization product effluent may include any suitable amounts of these hydrocarbons. For example, the depolymerization product effluent may include an amount of C2-C4 hydrocarbon in a range from about 65 wt % to about 98 wt %, such as from about 70 wt % to about 95 wt % such as from about 75 wt % to about 90 wt %, such as from about 80 wt % to about 90 wt %, such as about 85 wt % based on a total amount of C2-C4 hydrocarbon, C5-C11 hydrocarbon, C12+ hydrocarbon, and methane present in the depolymerization product effluent. The total amount of C2-C4 hydrocarbon, C5-C11 hydrocarbon, C12+ hydrocarbon, and methane present in the depolymerization product effluent is equal to 100 wt %.

The depolymerization product effluent may include an amount of C5-C11 hydrocarbon in a range from about 1 wt % to about 30 wt %, such as from about 5 wt % to about 25 wt %, such as from about 10 wt % to about 20 wt %, such as about 15 wt % based on the total amount of C2-C4 hydrocarbon, C5-C11 hydrocarbon, C12+ hydrocarbon, and methane present in the depolymerization product effluent.

The depolymerization product effluent may include an amount of the C12+ hydrocarbon in a range from about 1 wt % to about 20 wt %, such as from about 3 wt % to about 15 wt %, such as from about 4 wt % to about 10 wt %, such as about 5 wt % based on the total amount of C2-C4 hydrocarbon, C5-C11 hydrocarbon, C12+ hydrocarbon, and methane present in the depolymerization product effluent.

Methane may or may not be present in the depolymerization product effluent. If present, the depolymerization product effluent may include an amount of methane in a range from greater than 0 wt % to about 3 wt %, such as from greater than 0 wt % to about 2 wt %, or from about 1 wt % to about 3 wt % based on the total amount of C2-C4 hydrocarbon, C5-C11 hydrocarbon, C12+ hydrocarbon, and methane present in the depolymerization product effluent.

The amounts of these hydrocarbons may be controlled, if desired, depending on, for example, the waste plastic feed entering the process 100, the temperature at which the waste plastic feed is heated and/or other conditions at operation 105, the depolymerization catalyst utilized at operation 115, the depolymerization conditions at operation 115, combinations thereof, among other parameters.

The hydrocarbons present in the depolymerization product effluent may be separated into, for example, one or more, or a plurality of, output streams. Accordingly, the process 100 may further include separating the C12+ hydrocarbon from the depolymerization product effluent at operation 120. The C12+ hydrocarbon separated may include one or more C12-C50 hydrocarbons, such as one or more C20-50 hydrocarbons or one or more C12-C30 hydrocarbons. For example, the C12+ hydrocarbon may include VGO which includes C20-C50 hydrocarbons. As described herein, the C12+ hydrocarbon may include one or more C12+ paraffins, one or more C12+ olefins, one or more C12+ aromatics, or combinations thereof. The C12+ hydrocarbon separated at operation 120 may be referred to as a fraction or an effluent.

The separation process of operation 120 may include performing a gas-liquid separation of the depolymerization product effluent, a liquid-liquid separation of the depolymerization product effluent, or a combination thereof. Any suitable gas-liquid separation technique of the depolymerization product effluent and/or any suitable liquid-liquid separation technique of the depolymerization product effluent may be utilized. For example, the gas-liquid separation, the liquid-liquid separation, or the combination thereof may include performing a distillation, a flash an evaporation, a fractionation, an extraction, a decantation, a coalescence, or combinations thereof on the depolymerization product effluent.

The process 100 may further include separating the C2-C4 hydrocarbon from the depolymerization product effluent. The C2-C4 hydrocarbon may be separated from the depolymerization product effluent by, for example, a gas-liquid separation of the depolymerization product effluent. The C2-C4 hydrocarbon separated may be referred to as a fraction or an effluent. The process 100 may further include separating the C5-C11 hydrocarbon from the depolymerization product effluent. The C5-C11 hydrocarbon may be separated from the depolymerization product effluent by, for example, a gas-liquid separation of the depolymerization product effluent, a liquid-liquid separation of the depolymerization product effluent, or a combination thereof. The C5-C11 hydrocarbon separated may be referred to as a fraction or an effluent.

Any suitable gas-liquid separation techniques and liquid-liquid separation techniques may be utilized for separating the C2-C4 hydrocarbon and/or C5-C11 hydrocarbon from the depolymerization product effluent. For example, suitable gas-liquid separation techniques and liquid-liquid separation techniques may include distillation, flash evaporation, fractionation, extraction, decantation, coalescence, or combinations thereof, of the depolymerization product effluent. If methane is present in the depolymerization product effluent, the process 100 may further include separating the methane from the depolymerization product effluent by any suitable gas-liquid separation technique.

In some aspects, which may be combined with other aspects, one or more of the C2-C4 hydrocarbon, the C5-C11 hydrocarbon, the C12+ hydrocarbon, or the methane (if present) may be separated from the depolymerization product effluent concurrently or sequentially.

Referring back to FIG. 1, the process 100 may further include introducing the C12+ hydrocarbon to the dissolution unit at operation 125. Here, following separation of the C12+ hydrocarbon from the depolymerization product effluent, the C12+ hydrocarbon may be transferred to the dissolution unit. At the dissolution unit, the C12+ hydrocarbon may be heated with the waste plastic feed under suitable conditions such as those described herein with respect to operation 105.

In some aspects, which may be combined with other aspects, the C2-C4 hydrocarbon (which may include saturated or unsaturated hydrocarbons) may be processed or upgraded to, for example, one or more olefins. Here, the process 100 may further include producing olefins, such as C2-C4 olefins, from the C2-C4 hydrocarbon separated. As an example, the C2-C4 hydrocarbon separated may be fed to any suitable olefin producing unit, such as a steam cracker, a dehydrogenation unit, or a combination thereof, to produce C2-C4 olefins. In some aspects, which may be combined with other aspects, the C5-C11 hydrocarbon (which may include saturated or unsaturated hydrocarbons) may be processed or upgraded to, for example, one or more olefins. Here, the process 100 may further include producing olefins, such as C5-C11 olefins, from the C5-C11 hydrocarbon separated. As an example, the C5-C11 hydrocarbon separated may be fed to any suitable olefin producing unit, such as a steam cracker, a dehydrogenation unit, or a combination thereof, to produce C5-C11 olefins.

Aspects of the present disclosure also generally relate to waste plastic conversion plants. FIG. 2 is a generalized schematic flow diagram showing various implementations of processes described herein corresponding to operational areas or units in a waste plastic conversion plant 200. Aspects and implementations of the waste plastic conversion plant 200 may be combined with other aspects and implementations described herein, such as aspects and implementations of process 100. The waste plastic conversion plant, interchangeably referred to as a plant for converting waste plastic, serves to process waste plastic.

The waste plastic conversion plant 200 includes a feedstock unit 220 holding the waste plastic feed. If desired, mechanical treatment of the waste plastic feed, such as comminution of the waste plastic feed as described herein, may be performed at the feedstock unit 220. Accordingly, the feedstock unit may be configured to mechanically treat the waste plastic feed.

The waste plastic feed may exit the feedstock unit 220 and flow through line 201 to enter a dissolution unit 230. At the dissolution unit 230, operation 105 may be performed, whereby the waste plastic feed is heated with an optional dissolution medium, an optional C12+ hydrocarbon, or combinations thereof to form a mixture. The dissolution unit 230 may be configured to receive a first feed including a waste plastic. The dissolution unit may be further configured to receive a second feed including a dissolution medium. The dissolution medium may enter the dissolution unit 230 by line 210. The dissolution unit may be further configured to receive a first output stream from the second separation unit 260, the first output stream including the C12+ hydrocarbon. As described below, the first output stream is produced from the second separation unit 260. The first output stream that includes the C12+ hydrocarbon may enter the dissolution unit by line 207. The dissolution unit 230 may also be configured to heat the first feed, the second feed, the third feed, or combinations thereof to provide a mixture that includes an insoluble component and a soluble component.

The dissolution unit 230 may be further configured to mix one or more of the first feed that includes the waste plastic, the second feed that includes the dissolution medium, the first output stream that includes the C12+ hydrocarbon, or combinations thereof. To accomplish the mixing, the dissolution unit 230 may include a mixer, such as an impeller, that is configured to mix the first feed, the second feed, the first output stream, or combinations thereof.

Prior to entering the dissolution unit 230, at least a portion of water, at least a portion of O₂, or a combination thereof, if present in the waste plastic feed, may be removed from the waste plastic feed and exit the process through line 202. Illustrative, but non-limiting, techniques for removing water, O₂, or a combination thereof, are described herein and may include thermal drying, hydraulically pressing, centrifugal dewatering, purging with a non-reactive gas, or combinations thereof.

The mixture that includes the insoluble component and the soluble component may exit the dissolution unit 230 and flow through line 203 to enter a first separation unit 240. At the first separation unit 240, operation 110 may be performed, whereby the soluble component of the mixture is separated from the insoluble component of the mixture. The first separation unit 240 may be configured to receive the mixture from the dissolution unit 230. Here, the first separation unit 240 may include an inlet coupled to line 203, the inlet configured to receive the mixture from the dissolution unit 230.

The first separation unit 240 may be further configured to separate the mixture that includes the insoluble component and the soluble component. The insoluble component may exit the first separation unit 240 via line 204. The insoluble component may exit the first separation unit 240 by a first outlet of the first separation unit 240 coupled to line 204, the first outlet of the first separation unit 240 configured to discharge the insoluble component. The soluble component may exit the first separation unit 240 through line 205. The soluble component may exit the first separation unit 240 by a second outlet of the first separation unit 240 coupled to line 205, the second outlet of the first separation unit 240 configured to discharge the insoluble component.

The soluble component separated from the mixture at the first separation unit 240 may flow through the line 205 and enter a depolymerization reactor 250. At the depolymerization reactor 250, operation 115 may be performed, whereby the soluble component is contacted with a depolymerization catalyst to form the depolymerization product effluent. The depolymerization reactor 250 may be configured to receive the soluble component from the first separation unit 240. Here, the depolymerization reactor 250 may include a first inlet coupled to line 205, the first inlet of the depolymerization reactor 250 configured to receive the soluble component. The depolymerization reactor 250 may be further configured to contact the soluble component with a depolymerization catalyst under depolymerization conditions. The depolymerization reactor 250 may be further configured to discharge a depolymerization product effluent, such as the depolymerization product effluent described herein. Here, the depolymerization reactor 250 may include a first outlet coupled to line 206, the first outlet of the depolymerization reactor 250 configured to discharge the depolymerization product effluent.

In some aspects, which may be combined with other aspects, the depolymerization reactor 250 may further include an optional second outlet configured to discharge a spent depolymerization catalyst. The optional second outlet is coupled to line 211 through which the spent depolymerization catalyst may exit the depolymerization reactor 250. In some aspects, which may be combined with other aspects, the depolymerization reactor 250 may further include an optional second inlet configured to receive fresh depolymerization catalyst, depolymerization catalyst regenerated from the spent depolymerization catalyst, or combinations thereof. The optional second inlet is coupled to line 212 through which the fresh depolymerization catalyst, the depolymerization catalyst regenerated from the spent depolymerization catalyst, or combinations thereof, may enter the depolymerization reactor 250.

The depolymerization product effluent may exit the depolymerization reactor 250 and flow through the line 206 to enter a second separation unit 260. At the second separation unit 260, operation 120 may be performed, whereby the C12+ hydrocarbon is separated from the depolymerization product effluent. The second separation unit 260 may be configured to receive the depolymerization product effluent from the depolymerization reactor 250. Here, the second separation unit 260 may include an inlet coupled to line 206, the inlet of the second separation unit 260 configured to receive the depolymerization product effluent. The second separation unit 260 may be further configured to separate the depolymerization product effluent into a plurality of output streams. The plurality of output streams may include gaseous hydrocarbons, liquid hydrocarbons, or combinations thereof hydrocarbons. The second separation unit 260 may be further configured discharge the plurality of output streams.

A first output stream of the plurality of output streams includes the C12+ hydrocarbon. The first output stream including the C12+ hydrocarbon may exit the second separation unit 260 via the line 207. The line 207 is configured to transfer the first output stream that includes the C12+ hydrocarbon to the dissolution unit 230. For example, the line 207 is configured to perform operation 125, whereby the C12+ hydrocarbon is introduced to the dissolution unit 230. The line may include a pump or other suitable element to assist in transferring the C12+ hydrocarbon from the second separation unit 260 to the dissolution unit 230.

A second output stream of the plurality of output streams includes the C2-C4 hydrocarbon. The second output stream may exit the second separation unit 260 via the line 208. A third output stream of the plurality of output streams includes the C5-C11 hydrocarbon. The third output stream may exit the second separation unit 260 via the line 209. If methane is present in the depolymerization product effluent, the second separation unit 260 may be configured to discharge a fourth output stream that includes methane via line 213.

The waste plastic conversion plant 200 may optionally include a first olefin producing unit 270. The first olefin producing unit 270 may be configured to produce C2-C4 olefins. The first olefin producing unit 270 may be further configured to receive the second output stream that includes the C2-C4 hydrocarbon flowing through line 208. The first olefin producing unit 270 may be further configured to produce olefins from the second output stream that includes the C2-C4 hydrocarbon. The first olefin producing unit 270 may include any suitable apparatus for producing C2-C4 olefins such as a steam cracker, a dehydrogenation unit, or a combination thereof, though other apparatus are contemplated.

The waste plastic conversion plant 200 may optionally include second olefin producing unit 280. The second olefin producing unit 280 may be configured to produce C5-C11 olefins. The second olefin producing unit 280 may be further configured to receive the third output stream that includes the C5-C11 hydrocarbon flowing through line 209. The second olefin producing unit 280 may be further configured to produce olefins from the third output stream that includes the C5-C11 hydrocarbon. The second olefin producing unit 280 may include any suitable apparatus for producing C5-C11 olefins such as a steam cracker, a dehydrogenation unit, or a combination thereof, though other apparatus are contemplated.

Optionally one or more elements described with respect to the waste plastic conversion plant 200 may be coupled to a controller 290, as shown by the dashed lines in FIG. 2. The controller 290 may be utilized to control, for example, one or more operating parameters of the one or more elements illustrated in the waste plastic conversion plant 200, one or more operations of processes described herein (for example, one or more operations of process 100), or combinations thereof. The controller may include a processor, memory, and support circuits. The processor may be one of any form of general purpose microprocessor, or a general purpose central processing unit (CPU), each of which may be used in an industrial setting, such as a programmable logic controller (PLC), supervisory control and data acquisition (SCADA) systems, or other suitable industrial controller.

The memory is non-transitory and may be one or more of readily available memory such as random access memory (RAM), read only memory (ROM), or any other form of digital storage, local or remote. The memory contains instructions, that when executed by the processor, may facilitate the operation of one or more elements illustrated in FIG. 2, one or more operations of process 100, or combinations thereof. The instructions in the memory are in the form of a program product such as a program that implements the method of the present disclosure. The program code of the program product may conform to any one of a number of different programming languages. Illustrative computer-readable storage media include, but are not limited to: (i) non-writable storage media (for example, read-only memory devices within a computer such as CD-ROM disks readable by a CD-ROM drive, flash memory, ROM chips, or any type of solid-state non-volatile semiconductor memory) on which information is permanently stored; and (ii) writable storage media (for example, floppy disks within a diskette drive or hard-disk drive or any type of solid-state random-access semiconductor memory) on which alterable information is stored. Such computer-readable storage media, when carrying computer-readable instructions that direct the functions of the methods described herein, are examples of the present disclosure. The disclosure may be, for example, implemented as the program product stored on a computer-readable storage media (for example, memory) for use with a computer system (not shown). The program(s) of the program product define functions of the disclosure, described herein.

Although not shown in FIG. 2, it should be understood that suitable equipment for controlling, for example, temperature, pressure, and flow control of various feeds, effluents, and output streams may be used with the waste plastic conversion plant 200. For example, heat exchangers may be used to cool or heat a liquid or a gas along one or more lines or within various units or reactors of the waste plastic conversion plant 200. Pumps and motors can be utilized to control the rate of flow of the materials traveling or flowing through the lines and the operating pressures of various components of the waste plastic conversion plant 200. Further, the waste plastic conversion plant 200 may include valves or other release mechanisms for, e.g., purging gases or liquids from the system. Various process controls may be used. Such process controls can include probes and sensors such as pressure indicators, differential pressure cells, temperature indicators, thermocouples, temperature switches, resistance temperature detectors, solenoids, flowmeters, flow regulators and valves, gas analyzers, humidity sensors, radar sensors, ammeters, current meters, liquid level detectors, feed level probes, electrical drives, and combinations thereof.

Aspects of the present disclosure generally relate to new processes for converting waste plastic. Aspects of the present disclosure also generally relate to waste plastic conversion plants. A C12+ hydrocarbon produced by aspects described herein may be re-used in the process. Here, the C12+ hydrocarbon may be introduced to the process to aid in dissolution of the waste plastic feed. A C2-C4 hydrocarbon produced by aspects described herein may be used as a feedstock for producing olefins. A C5-C11 hydrocarbon produced by aspects described herein may be used as a feedstock for producing olefins. In contrast to conventional technologies, aspects described herein eliminate the need to purify waste plastic prior to depolymerization. Additionally, relative to output streams produced by state-of-the-art technologies that are characterized as having contamination and abated materials properties, aspects described herein may be utilized to limit side reactions, to produce hydrocarbon output streams having improved properties, or both.

### ASPECTS OF THE DISCLOSURE

The present disclosure provides, among others, the following aspects, each of which may be considered as optionally including any alternate aspects:

Aspect 1. A process for converting waste plastic, the process comprising:
heating, in a dissolution unit, a waste plastic feed with a dissolution medium to form a mixture comprising an insoluble component and a soluble component;
separating the soluble component of the mixture from the insoluble component of the mixture;
contacting the soluble component with a depolymerization catalyst under depolymerization conditions to form a depolymerization product effluent;
separating a C12+ hydrocarbon (for example, a C12-C50 hydrocarbon, such as a C20-50 hydrocarbon or a C12-C30 hydrocarbon) from the depolymerization product effluent; and
introducing the C12+ hydrocarbon to the dissolution unit to heat with the waste plastic feed.

Aspect 2. The process according to Aspect 1, wherein the C12+ hydrocarbon introduced to the dissolution unit comprises a C12+ paraffin, a C12+ olefin, a C12+ aromatic, or combinations thereof.

Aspect 3. The process according to any one of the preceding Aspects, wherein, prior to the heating the waste plastic feed with the dissolution medium, the process further comprises:
removing at least a portion of water, at least a portion of O₂, or a combination thereof from the waste plastic feed such that the waste plastic feed comprises:
   about 5,000 ppm or less water, based on a total weight of the waste plastic feed;
   about 5,000 ppm or less O₂ based on the total weight of the waste plastic feed; or
   a combination thereof.

Aspect 4. The process according to any one of the preceding Aspects, wherein the heating the waste plastic feed with the dissolution medium comprises selectively dissolving at least a portion of the waste plastic feed at:
a temperature that is about 250° C. or less, such as in a range from about 75° C. to about 225° C., such as from about 110° C. to about 200° C., such as from about 130° C. to about 180° C., such as from about 140° C. to about 160° C.;
a pressure in a range from about 100 kPa to about 2,000 kPa, such as from about 500 kPa to about 1,500 kPa, such as from about 750 kPa to about 1,250 kPa; or
combinations thereof.

Aspect 5. The process according to any one of the preceding Aspects, wherein the heating the waste plastic feed with the dissolution medium is performed in a continuous stirred tank reactor.

Aspect 6. The process according to any one of the preceding Aspects, wherein the waste plastic feed comprises a polyolefin.

Aspect 7. The process according to Aspect 6, wherein the polyolefin comprises polyethylene (for example, high density polyethylene, medium density polyethylene, low density polyethylene, or linear low density polyethylene), polypropylene (for example, a polypropylene random block copolymer), polystyrene, or combinations thereof.

Aspect 8. The process according to any one of Aspects 6 or 7, wherein the polyolefin comprises an isotactic polyolefin, an atactic polyolefin, a syndiotactic polyolefin, or combinations thereof.

Aspect 9. The process according to any one of Aspects 6-8, wherein the polyolefin comprises a polyolefin homopolymer, a polyolefin copolymer, a polyolefin block copolymer, a polyolefin random block copolymer, or combinations thereof.

Aspect 10. The process according to any one of Aspects 6-9, wherein the waste plastic feed comprises an amount of the polyolefin of about 10 wt % or more, such as in a range from about 10 wt % to about 99 wt %, such as from about 20 wt % to about 98 wt %, such as from about 40 wt % to about 95 wt %, such as from about 50 wt % to about 90 wt %, such as from about 70 wt % to about 85 wt % based on a total weight of the waste plastic feed.

Aspect 11. The process according to any one of Aspects 6-10, wherein the waste plastic feed further comprises a waste plastic other than the polyolefin.

Aspect 12. The process according to Aspect 11, wherein the waste plastic other than the polyolefin comprises polyester (for example, polyethylene terephthalate, polybutylene terephthalate, or a combination thereof), polyamide, polyurethane, polyphenol, polycarbonate, a halogen-containing polymer, polylactic acid, polyacrylic acid, polyacrylate, polyacetal, or combinations thereof.

Aspect 13. The process according to Aspect 12, wherein, when the waste plastic feed comprises the halogen-containing polymer, the halogen-containing polymer comprises a chlorinated polymer (for example, chlorinated polyethylene), polyvinylchloride, polyvinylidene chloride, a fluorinated polymer (for example, fluorinated polyethylene, such as polytetrafluoroethylene), or combinations thereof.

Aspect 14. The process according to any one of Aspects 11-13, wherein the waste plastic feed comprises an amount of the waste plastic other than the polyolefin that is about 10 wt % or less, such as in a range from about 1 wt % to about 10 wt %, such as from about 2 wt % to about 5 wt %, based on a total weight of the waste plastic feed

Aspect 15. The process according to any one of the preceding Aspects, wherein the waste plastic feed comprises a halogen content, if present, in an amount of about 36,000 ppm or less, such as about 10,000 ppm or less, or in a range from greater than 0 ppm to about 36,000 ppm, such as from greater than 0 ppm to about 10,000 ppm based on a total weight of the waste plastic feed.

Aspect 16. The process according to any one of the preceding Aspects, wherein the waste plastic feed further comprises a virgin plastic.

Aspect 17. The process according to any one of the preceding Aspects, wherein the waste plastic feed comprises:
a solid characterized by an average particle size of about 10 cm or less, such as about 1 cm or less, such as about 100 mm or less, such as about 10 mm or less, such as about 5 mm or less, such as about 3 mm or less, such as about 1 mm or less;
a solid in pellet form and characterized by an average particle size in a range from about 0.01 mm to about 10 mm, such as from about 0.1 mm to about 1 mm;
a solid in fluff form and characterized by an average particle size of about 1,500 μm or less, such as about 1,000 μm or less, such as about 500 μm or less, such as about 400 μm or less, such as about 300 μm or less, such as about 100 μm or less, such as about 50 μm or less, such as about 10 μm or less; or
combinations thereof.

Aspect 18. The process according to any one of the preceding Aspects, wherein the insoluble component of the mixture comprises a rubber (for example, nitrile rubber), a filler (for example, calcium carbonate, talk, carbon black, or glass fiber), a pigment (for example, titanium dioxide), a dye, a process aid, a stabilizer, an ink, a plasticizer, a slip agent, an antiblock agent, an antioxidant, a UV stabilizer, a cellulose fiber, an adhesive, or combinations thereof.

Aspect 19. The process according to any one of the preceding Aspects, wherein the heating the waste plastic feed with the dissolution medium is:
performed under conditions effective to dissolve polyolefin(s) present in the waste plastic feed;
performed under conditions that minimize dissolution of a halogen-containing polymer, such as chlorinated polymer (for example, chlorinated polyethylene), polyvinylchloride, polyvinylidene chloride, a fluorinated polymer, or combinations thereof, present in the waste plastic feed; or
a combination thereof.

Aspect 20. The process according to any one of the preceding Aspects, wherein the insoluble component comprises a waste plastic other than a polyolefin, such as a halogen-containing polymer.

Aspect 21. The process according to any one of the preceding Aspects, wherein the dissolution medium comprises a hydrocarbon feed.

Aspect 22. The process according to Aspect 21, wherein:
the hydrocarbon feed has a boiling point in a range from about 214° C. to about 680° C.;
the hydrocarbon feed comprises a petroleum-based material (for example, vacuum gas oil (VGO)), a fossil fuel-based material, a bio-based material, or combinations thereof;
or combinations thereof.

Aspect 23. The process according to any one of the preceding Aspects, wherein the separating the soluble component of the mixture from the insoluble component of the mixture comprises filtration, vacuum filtration, centrifugation, decantation, decanting centrifugation, or combinations thereof, among other techniques.

Aspect 24. The process according to any one of the preceding Aspects, wherein the depolymerization catalyst comprises a zeolite-based catalyst, a chromia-based catalyst, a non-zeolite cracking catalyst, a solid acid catalyst, or combinations thereof.

Aspect 25. The process according to Aspect 24, wherein the zeolite-based catalyst, the chromia-based catalyst, the non-zeolite cracking catalyst, or the combinations thereof achieves dehydrogenation, hydrogenolysis, cracking, isomerization, dehydrohalogenation, dehalogenation, or a combination thereof, of hydrocarbons present in the soluble component.

Aspect 26. The process according to any one of Aspects 24 or 25, wherein the depolymerization catalyst comprises the zeolite-based catalyst.

Aspect 27. The process according to Aspect 26, wherein the zeolite-based catalyst comprises L-zeolite (zeolite L or LTL), X-zeolite (zeolite X), Y-zeolite (Zeolite Y), omega zeolite, beta zeolite, SAPO-34 zeolite, USY Zeolite, HY zeolite, ZSM-4, ZSM-5 (MFI), ZSM-10, ZSM-11, ZSM-12, ZSM-20, ZSM-22, ZSM-23, ZSM-34, ZSM-35, ZSM-50, REY, USY, RE-USY, LZ-210, LZ-210-A, LZ-210-M, LZ-210-T, SSZ-13, SSZ-24, SSZ-26, SSZ-31, SSZ-33, SSZ-35, SSZ-37, SSZ-41, SSZ-42, SSZ-44, MCM-58, H-MOR (H-mordenite), mazzite, faujasite, chabazite, a modified mesoporous form thereof, an acid-modified form thereof, or combinations thereof.

Aspect 28. The process according to any one of Aspects 26 or 27, wherein the zeolite-based catalyst comprises a transition metal comprising chromium, molybdenum, tungsten, iron, ruthenium, osmium, cobalt, rhodium, iridium, nickel, palladium, platinum, gold, zinc, titanium, tantalum, or combinations thereof, such as ruthenium, osmium, rhodium, iridium, palladium, platinum, gold, or combinations thereof.

Aspect 29. The process according to Aspect 28, wherein the zeolite-based catalyst comprises a binder comprising an inorganic oxide such as silica, alumina, silica-alumina, clay, titania, magnesium oxide, or combinations thereof.

Aspect 30. The process according to any one of Aspects 26-29, wherein the zeolite-based catalyst comprises Pt-L-zeolite, Pt-ZSM-5, Pt-Y zeolite, Pt-SAPO-34 zeolite, Pt-USY zeolite, Pt-HY zeolite, Pt-beta zeolite, or combinations thereof.

Aspect 31. The process according to any one of Aspects 26-30, wherein the zeolite-based catalyst further comprises a promoter comprising a Group 1 metal, a Group 2 metal, a Group 14 metal, a Group 15 metal, a Group 15 non-metal, or combinations thereof

Aspect 32. The process according to Aspect 31, wherein the promoter comprises tin, bismuth, phosphorous, or combinations thereof.

Aspect 33. The process according to Aspect 32, wherein the zeolite-based catalyst is treated with a tin salt, such as stannous chloride, stannic chloride, stannic tartrate, stannic nitrate, or combinations thereof.

Aspect 34. The process according to any one of Aspects 26-33, wherein the zeolite of the zeolite-based catalyst is characterized by a zeolite pore volume in a range from about 0.10 mL/g to about 2.0 mL/g, such as from about 0.25 mL/g to about 1.75 mL/g, such as from about 0.5 mL/g to about 1.5 mL/g, such as from about 0.75 mL/g to about 1.25 mL/g, such as about 1.0 mL/g.

Aspect 35. The process according to any one of Aspects 26-34, wherein the zeolite of the zeolite-based catalyst is characterized by a zeolite pore diameter in a range from about 3.0 Å to about 10.0 Å, such as from about 4.0 Å to about 9.0 Å, such as from about 5.0 to about 8.0, such as from about 5.5 Å to about 7.0 Å.

Aspect 36. The process according to any one of Aspects 26-35, wherein the zeolite of the zeolite-based catalyst is characterized by a zeolite pore diameter in a range from about 7.0 Å to about 10.0 Å, or from about 5.0 Å to about 6.0 Å, or from about 2.0 Å to about 3.0 Å.

Aspect 37. The process according to any one of Aspects 26-36, wherein the zeolite-based catalyst is characterized by a catalyst surface area in a range from about 100 m²/g to about 1,000 m²/g, such as from about 200 m²/g to about 900 m²/g, such as from about 300 m²/g to about 800 m²/g, such as from about 400 to about 700 m²/g, such as from about 500 m²/g to about 600 m²/g.

Aspect 38. The process according to any one of Aspects 26-37, wherein the zeolite-based catalyst is in particulate form having an average particle size in a range from about 1 μm to about 100 μm, such as from about 2 μm to about 50 μm, such as from about 3 μm to about 30 μm, such as from about 4 μm to about 20 μm, or from about 5 μm to about 20 μm.

Aspect 39. The process according to any one of Aspects 26-38, wherein the zeolite-based catalyst comprises a Y-zeolite and is in particulate form having an average particle size in a range from about 3 μm to about 8 μm.

Aspect 40. The process according to any one of Aspects 26-39, wherein the zeolite-based catalyst comprises ZSM-5 (MFI) and is in particulate form having an average particle size in a range from about 5 μm to about 10 μm.

Aspect 41. The process according to any one of Aspects 26-40, wherein the zeolite-based catalyst comprises H-MOR and is in particulate form having an average particle size in a range from about 12 μm to about 18 μm.

Aspect 42. The process according to any one of Aspects 26-41, wherein the zeolite-based catalyst comprises beta zeolite and is in particulate form having an average particle size in a range from about 3 μm to about 5 μm.

Aspect 43. The process according to any one of Aspects 24 or 25, wherein the depolymerization catalyst comprises the chromia-based catalyst.

Aspect 44. The process according to Aspect 43, wherein the chromia-based catalyst comprises an organic chromium compound (for example, chromium acetylacetonate), amorphous Cr₂O₃, crystalline Cr₂O₃, or combinations thereof.

Aspect 45. The process according to any one of Aspects 43 or 44, wherein the chromia-based catalyst is supported on silica, silica-alumina, silica-coated alumina, silica-titania, silica-magnesia, alumina, zirconia, thoria, mixed oxides thereof, or combinations thereof.

Aspect 46. The process according to any one of Aspects 43-45, wherein the chromia-based catalyst comprises: chromium acetylacetonate; chromia-alumina; chromia-magnesia-alumina; magnesium chromite-tin oxide; magnesium chromite-alumina-tin oxide; magnesium chromite combined with a promoter comprising one or more of B, Si, Sn, Pb, Zn, or Se; or combinations thereof.

Aspect 47. The process according to any one of aspects 24 or 25, wherein the depolymerization catalyst comprises the non-zeolite cracking catalyst.

Aspect 48. The process according to Aspect 47, wherein the non-zeolite cracking catalyst comprises clay, acid treated clay, perovskite, layered titanate, silica alumina, silica-coated alumina, acidic alumina, tungstated zirconia, activated carbon, natural kaolin, acid-modified kaolin, bentonite, or combinations thereof.

Aspect 49. The process according to any one of Aspects 24 or 25, wherein the depolymerization catalyst comprises the solid acid catalyst, the solid acid catalyst comprising a solid oxide treated with an electron withdrawing anion.

Aspect 50. The process according to Aspect 49, wherein the solid oxide treated with the electron withdrawing anion comprises alumina, silica-alumina, silica-coated alumina, silica-zirconia, silica-titania, or combinations thereof.

Aspect 51. The process according to any one of Aspects 49 or 50, wherein the solid oxide treated with the electron withdrawing anion comprises a sulfated solid oxide, a fluorided solid oxide, a phosphated solid oxide, a fluorophosphated solid oxide, or a combinations thereof.

Aspect 52. The process according to any one of Aspects 49-51, wherein the solid oxide treated with the electron withdrawing anion comprises fluorided alumina, chlorided alumina, bromided alumina, phosphated alumina, sulfated alumina, fluorided silica-alumina, chlorided silica-alumina, bromided silica-alumina, sulfated silica-alumina, fluorided silica-coated alumina, sulfated silica-coated alumina, phosphated silica-coated alumina, fluorophosphated silica-coated alumina, fluorided silica-titania, fluorided silica-zirconia, chlorided silica-zirconia, bromided silica-zirconia, sulfated silica-zirconia, fluorided silica-zirconia, or combinations thereof.

Aspect 53. The process according to Aspect 52, wherein the solid oxide treated with the electron withdrawing anion comprises the fluorided alumina, the sulfated alumina, the fluorided silica-alumina, the sulfated silica-alumina, the fluorided silica-coated alumina, the phosphated silica-coated alumina, the sulfated silica-coated alumina, or combinations thereof.

Aspect 54. The process according to any one of the preceding Aspects, wherein the depolymerization conditions comprise a depolymerization temperature in a range from about 200° C. to about 800° C., such as from about 300° C. to about 700° C., such as from about 400° C. to about 600° C., such as from about 450° C. to about 550° C., or in a range from about 200° C. to about 300° C., such as from about 200° C. to about 250° C., while contacting the soluble component with the depolymerization catalyst.

Aspect 55. The process according to any one of the preceding Aspects, wherein the depolymerization conditions comprise a duration sufficient to depolymerize at least a portion of hydrocarbons present in the soluble component.

Aspect 56. The process according to any one of the preceding Aspects, wherein the depolymerization conditions comprise contacting the soluble component with the depolymerization catalyst in the presence of hydrogen at a pressure in a range from about 34 kPa to about 6,895 kPa, such as from about 335 kPa to about 5,516 kPa, such as from about 689 kPa to about 2,069 kPa, such as from about 1,034 kPa to about 1,724 kPa.

Aspect 57. The process according to any one of the preceding Aspects, wherein the contacting the soluble component with the depolymerization catalyst is performed in a fixed bed reactor or a fluidized bed reactor.

Aspect 58. The process according to any one of the preceding Aspects, wherein the separating the C12+ hydrocarbon from the depolymerization product effluent comprises performing a gas-liquid separation of the depolymerization product effluent, a liquid-liquid separation of the depolymerization product effluent, or a combination thereof.

Aspect 59. The process according to Aspect 58, wherein the performing the gas-liquid separation, the liquid-liquid separation, or the combination thereof comprises distillation, flash evaporation, fractionation, extraction, decantation, coalescence, or combinations thereof.

Aspect 60. The process according to any one of the preceding Aspects, wherein the depolymerization product effluent further comprises: a C2-C4 hydrocarbon; and a C5-C11 hydrocarbon (for example, naphtha).

Aspect 61. The process according to any one of the preceding Aspects, wherein the depolymerization product effluent further comprises methane.

Aspect 62. The process according to any one of the preceding Aspects, wherein the depolymerization product effluent comprises:
an amount of C2-C4 hydrocarbon in a range from about 65 wt % to about 98 wt %, such as from about 70 wt % to about 95 wt % such as from about 75 wt % to about 90 wt %, such as from about 80 wt % to about 90 wt %, such as about 85 wt % based on a total amount of C2-C4 hydrocarbon, C5-C11 hydrocarbon, C12+ hydrocarbon, and methane present in the depolymerization product effluent, the total amount of C2-C4 hydrocarbon, C5-C11 hydrocarbon, C12+ hydrocarbon, and methane present in the depolymerization product effluent equal to 100 wt %;
an amount of C5-C11 hydrocarbon in a range from about 1 wt % to about 30 wt %, such as from about 5 wt % to about 25 wt %, such as from about 10 wt % to about 20 wt %, such as about 15 wt % based on the total amount of C2-C4 hydrocarbon, C5-C11 hydrocarbon, C12+ hydrocarbon, and methane present in the depolymerization product effluent;
an amount of the C12+ hydrocarbon in a range from about 1 wt % to about 20 wt %, such as from about 3 wt % to about 15 wt %, such as from about 4 wt % to about 10 wt %, such as about 5 wt % based on the total amount of C2-C4 hydrocarbon, C5-C11 hydrocarbon, C12+ hydrocarbon, and methane present in the depolymerization product effluent; and
an amount of methane, if present, in a range from greater than 0 wt % to about 3 wt %, such as from greater than 0 wt % to about 2 wt % or from about 1 wt % to about 3 wt % based on the total amount of C2-C4 hydrocarbon, C5-C11 hydrocarbon, C12+ hydrocarbon, and methane present in the depolymerization product effluent.

Aspect 63. The process according to any one of Aspects 60-62, further comprising:
separating the C2-C4 hydrocarbon from the depolymerization product effluent by a gas-liquid separation of the depolymerization product effluent;
separating the C5-C11 hydrocarbon from the depolymerization product effluent by a gas-liquid separation of the depolymerization product effluent, a liquid-liquid separation of the depolymerization product effluent, or a combination thereof;
or a combination thereof.

Aspect 64. The process according to Aspect 63, wherein:
the C2-C4 hydrocarbon and C5-C11 hydrocarbon are separated from the depolymerization product effluent concurrently or sequentially;
one or more of the C2-C4 hydrocarbon, the C5-C11 hydrocarbon, the C12+ hydrocarbon, or the methane (if present) are separated from the depolymerization product effluent concurrently or sequentially; or
a combination thereof.

Aspect 65. The process according to any one of Aspects 60-64, further comprising producing olefins from the C2-C4 hydrocarbon separated from the depolymerization product effluent (for example, in a steam cracker, in a dehydrogenation unit, or a combination thereof).

Aspect 66. The process according to any one of Aspects 60-65, further comprising producing olefins from the C5-C11 hydrocarbon separated from the depolymerization product effluent (for example, in a steam cracker, in a dehydrogenation unit, or a combination thereof).

Aspect 67. The process according to any one of the preceding Aspects, wherein, prior to the heating the waste plastic feed with the dissolution medium, the process further comprises: comminuting the waste plastic feed.

Aspect 68. The process according to Aspect 67, wherein the comminuting the waste plastic feed comprises comminuting the waste plastic feed with a pelletizer, a shredder, a hammer mill, a grinder, a granulator, or combinations thereof.

Aspect 69. A process for converting waste plastic to hydrocarbons, the process comprising:
heating, under dissolution conditions and in a dissolution unit, a waste plastic feed with a dissolution medium to form a dissolution product, the dissolution conditions comprising a temperature in a range from about 100° C. to about 200° C.;
performing a solid-liquid separation on the dissolution product to separate a soluble component of the dissolution product from an insoluble component of the dissolution product;
contacting, under depolymerization conditions, hydrocarbon polymers (for example, C1-C50 hydrocarbon polymers, such as C2-C30 hydrocarbon polymers) present in the soluble component with a depolymerization catalyst to form a depolymerization product effluent;
separating the depolymerization product effluent into at least three fractions comprising: a first fraction comprising a C2-C4 hydrocarbon; a second fraction comprising a C5-11 hydrocarbon; and a third fraction comprising a C12+ hydrocarbon; and
combining the third fraction with the waste plastic feed in the dissolution unit.

Aspect 70. The process according to Aspect 69, wherein the insoluble component comprises solids, additives, heavy hydrocarbons (for example, C30-C50 hydrocarbons (which may be waxes)), halogen-containing polymers, or combinations thereof.

Aspect 71. The process according to any one of Aspects 69 or 70, wherein the performing the solid-liquid separation removes halogenated and/or oxygenated compounds, if present, in the waste plastic feed.

Aspect 72. The process according to any one of Aspects 69-71, wherein, prior to the heating the waste plastic feed with the dissolution medium, the process further comprises: removing at least a portion of water, oxygen, or combinations thereof from the waste plastic feed.

Aspect 73. The process according to any one of Aspects 69-72, wherein, prior to the heating the waste plastic feed with the dissolution medium, the process further comprises: comminuting the waste plastic feed.

Aspect 74. The process according to Aspect 74, wherein the comminuting the waste plastic feed comprises comminuting the waste plastic feed with a pelletizer, a shredder, a hammer mill, a grinder, a granulator, or combinations thereof.

Aspect 75. A plant for converting waste plastic, the plant comprising:
a dissolution unit configured to receive a first feed comprising waste plastic, to receive a second feed comprising a dissolution medium, to heat the first feed to provide a mixture comprising an insoluble component and a soluble component, to heat the first feed and second feed to provide a mixture comprising an insoluble component and a soluble component, or combinations thereof;
a first separation unit configured to receive the mixture from the dissolution unit and to separate the mixture;
a depolymerization reactor configured to receive the soluble component from the first separation unit, to contact the soluble component with a depolymerization catalyst under depolymerization conditions, to discharge a depolymerization product effluent, or combinations thereof;
a second separation unit configured to receive the depolymerization product effluent from the depolymerization reactor and to separate the depolymerization product effluent into a plurality of output streams; and
a line configured to transfer a first output stream of the plurality of output streams to the dissolution unit, the first output stream comprising a C12+ hydrocarbon.

Aspect 76. The plant according to Aspect 75, wherein the dissolution unit comprises a mixer, such as an impeller, configured to mix the first feed and the second feed.

Aspect 77. The plant according to any one of Aspects 75 or 76, wherein the mixer is further configured to mix the first feed with the first output stream.

Aspect 78. The plant according to any one of Aspects 75-77, wherein the first separation unit comprises: an inlet configured to receive the mixture from the dissolution unit; a first outlet configured to discharge the insoluble component; a second outlet configured to discharge the soluble component; or a combination thereof.

Aspect 79. The plant according to any one of Aspects 75-78, wherein the depolymerization reactor comprises: a first inlet configured to receive the soluble component; a first outlet configured to discharge the depolymerization product effluent; or a combination thereof.

Aspect 80. The plant according to Aspect 79, wherein the depolymerization reactor further comprises a second outlet configured to discharge a spent depolymerization catalyst.

Aspect 81. The plant according to any one of Aspects 75-80, wherein the depolymerization reactor comprises a catalyst inlet configured to receive fresh depolymerization catalyst, regenerated depolymerization catalyst, or combinations thereof.

Aspect 82. The plant according to any one of Aspects 75-81, wherein the second separation unit is configured to separate the depolymerization product effluent into gaseous hydrocarbons and liquid hydrocarbons.

Aspect 83. The plant according to any one of Aspects 75-82, wherein the second separation unit is configured to separate the depolymerization product effluent into: the first output stream comprising the C12+ hydrocarbon; a second output stream comprising a C2-C4 hydrocarbon; and a third output stream comprising a C5-C11 hydrocarbon.

Aspect 84. The plant according to Aspect 83, further comprising: a C2-C4 olefin producing unit configured to receive the second output stream and to produce a C2-C4 olefin from the second output stream; a C5-C11 olefin producing unit configured to receive the third output stream and to produce a C5-C11 olefin from the third output stream; or a combination thereof.

Aspect 85. The plant according to Aspect 84, wherein the C2-C4 olefin producing unit comprises a steam cracker, a dehydrogenation unit, or a combination thereof.

Aspect 86. The plant according to any one of Aspects 84 or 85, wherein the C5-C11 olefin producing unit comprises a steam cracker, a dehydrogenation unit, or a combination thereof.

Aspect 87. A process for converting waste plastic, the process comprising: heating, in a dissolution unit, a waste plastic feed with a dissolution medium to form a mixture comprising an insoluble component and a soluble component; separating the soluble component of the mixture from the insoluble component of the mixture; contacting the soluble component with a depolymerization catalyst under depolymerization conditions to form a depolymerization product effluent; separating a C12+ hydrocarbon from the depolymerization product effluent; and introducing the C12+ hydrocarbon to the dissolution unit to heat with the waste plastic feed.

Aspect 88. The process according to Aspect 87, wherein, prior to the heating the waste plastic feed with the dissolution medium, the process further comprises: removing at least a portion of water from the waste plastic feed; removing at least a portion of O₂ from the waste plastic feed; or a combination thereof.

Aspect 89. The process according to any one of Aspects 87 or 88, wherein the heating the waste plastic feed with the dissolution medium comprises selectively dissolving at least a portion of the waste plastic feed at a temperature that is about 250° C. or less.

Aspect 90. The process according to any one of Aspects 87-89, wherein the waste plastic feed comprises a polyolefin.

Aspect 91. The process according to Aspect 90, wherein the waste plastic feed further comprises a waste plastic other than the polyolefin in an amount that is about 10 wt % or less based on a total weight of the waste plastic feed.

Aspect 92. The process according to any one of Aspects 90 or 91, wherein the waste plastic other than the polyolefin comprises a polyester, a polyamide, a polyurethane, a polyphenol, a polycarbonate, a halogen-containing polymer, a polylactic acid, a polyacrylic acid, a polyacrylate, a polyacetal, or combinations thereof.

Aspect 93. The process according to any one of Aspects 87-92, wherein the waste plastic feed comprises a halogen content in an amount of about 36,000 ppm or less based on a total weight of the waste plastic feed.

Aspect 94. The process according to any one of Aspects 87-93, wherein: the insoluble component comprises a waste plastic other than a polyolefin; the insoluble component of the mixture comprises a rubber, a filler, a pigment, a dye, a process aid, a stabilizer, an ink, a plasticizer, a slip agent, an antiblock agent, an antioxidant, a UV stabilizer, a cellulose fiber, an adhesive, or combinations thereof; or a combination thereof.

Aspect 95. The process according to any one of Aspects 87-94, wherein the dissolution medium comprises: a hydrocarbon feed having a boiling point in a range from about 214° C. to about 680° C.; a hydrocarbon feed comprising a petroleum-based material, a fossil fuel-based material, a bio-based material, or combinations thereof; or a combination thereof.

Aspect 96. The process according to any one of Aspects 87-95, wherein the depolymerization catalyst comprises a zeolite-based catalyst, a chromia-based catalyst, a non-zeolite cracking catalyst, a solid acid catalyst, or combinations thereof.

Aspect 97. The process according to Aspect 95, wherein: when the depolymerization catalyst comprises the zeolite-based catalyst, the zeolite-based catalyst comprises L-zeolite (zeolite L or LTL), X-zeolite (zeolite X), Y-zeolite (Zeolite Y), omega zeolite, beta zeolite, SAPO-34 zeolite, USY Zeolite, HY zeolite, ZSM-4, ZSM-5 (MFI), ZSM-10, ZSM-11, ZSM-12, ZSM-20, ZSM-22, ZSM-23, ZSM-34, ZSM-35, ZSM-50, REY, USY, RE-USY, LZ-210, LZ-210-A, LZ-210-M, LZ-210-T, SSZ-13, SSZ-24, SSZ-26, SSZ-31, SSZ-33, SSZ-35, SSZ-37, SSZ-41, SSZ-42, SSZ-44, MCM-58, H-MOR (H-mordenite), mazzite, faujasite, chabazite, a modified mesoporous form thereof, an acid-modified form thereof, or combinations thereof; when the depolymerization catalyst comprises the chromia-based catalyst, the chromia-based catalyst comprises an organic chromium compound, amorphous Cr₂O₃, crystalline Cr₂O₃, or combinations thereof; when the depolymerization catalyst comprises the non-zeolite cracking catalyst, the non-zeolite cracking catalyst comprises clay, acid treated clay, perovskite, layered titanate, silica alumina, silica-coated alumina, acidic alumina, tungstated zirconia, activated carbon, natural kaolin, acid-modified kaolin, bentonite, or combinations thereof; when the depolymerization catalyst comprises the solid acid catalyst, the solid acid catalyst comprises a solid oxide treated with an electron withdrawing anion; or combinations thereof.

Aspect 98. The process according to any one of Aspects 87-97, wherein the depolymerization conditions comprise: heating at a depolymerization temperature in a range from about 200° C. to about 300° C. while contacting the soluble component with the depolymerization catalyst; contacting the soluble component with the depolymerization catalyst in the presence of hydrogen at a pressure in a range from about 689 kPa to about 2,069 kPa; or a combination thereof.

Aspect 99. The process according to any one of Aspects 87-98, wherein the depolymerization product effluent comprises: an amount of C2-C4 hydrocarbon in a range from about 65 wt % to about 98 wt % based on a total amount of C2-C4 hydrocarbon, C5-C11 hydrocarbon, C12+ hydrocarbon, and methane present in the depolymerization product effluent, the total amount of C2-C4 hydrocarbon, C5-C11 hydrocarbon, C12+ hydrocarbon, and methane present in the depolymerization product effluent equal to 100 wt %; an amount of C5-C11 hydrocarbon in a range from about 1 wt % to about 30 wt % based on the total amount of C2-C4 hydrocarbon, C5-C11 hydrocarbon, C12+ hydrocarbon, and methane present in the depolymerization product effluent; an amount of the C12+ hydrocarbon in a range from about 1 wt % to about 20 wt % based on the total amount of C2-C4 hydrocarbon, C5-C11 hydrocarbon, C12+ hydrocarbon, and methane present in the depolymerization product effluent; and an amount of methane, if present, in a range from greater than 0 wt % to about 3 wt % based on the total amount of C2-C4 hydrocarbon, C5-C11 hydrocarbon, C12+ hydrocarbon, and methane present in the depolymerization product effluent.

Aspect 100. The process according to any one of Aspects 87-99, further comprising: separating a C2-C4 hydrocarbon from the depolymerization product effluent; and separating a C5-C11 hydrocarbon from the depolymerization product effluent.

Aspect 101. The process according to Aspect 100, further comprising: producing olefins from the C2-C4 hydrocarbon separated from the depolymerization product effluent; producing olefins from the C5-C11 hydrocarbon separated from the depolymerization product effluent; or a combination thereof.

Aspect 102. A process for converting waste plastic to hydrocarbons, the process comprising: heating, under dissolution conditions and in a dissolution unit, a waste plastic feed with a dissolution medium to form a dissolution product, the dissolution conditions comprising a temperature in a range from about 100° C. to about 200° C.; performing a solid-liquid separation on the dissolution product to separate a soluble component of the dissolution product from an insoluble component of the dissolution product; contacting, under depolymerization conditions, hydrocarbon polymers present in the soluble component with a depolymerization catalyst to form a depolymerization product effluent; and separating the depolymerization product effluent into at least three fractions comprising: a first fraction comprising a C2-C4 hydrocarbon; a second fraction comprising a C5-11 hydrocarbon; and a third fraction comprising a C12+ hydrocarbon; and combining the third fraction with the waste plastic feed in the dissolution unit.

Aspect 103. The process of any one of Aspects 87-102, further comprising one or more of Aspects 1-74.

Aspect 103. A plant for converting waste plastic, the plant comprising: a dissolution unit configured to heat a feed comprising waste plastic to provide a mixture comprising an insoluble component and a soluble component; a first separation unit configured to receive the mixture from the dissolution unit and to separate the mixture; a depolymerization reactor configured to receive the soluble component from the first separation unit and to contact the soluble component with a depolymerization catalyst under depolymerization conditions to form a depolymerization product effluent; a second separation unit configured to receive the depolymerization product effluent from the depolymerization reactor and to separate the depolymerization product effluent into a plurality of output streams; and a line configured to transfer a first output stream of the plurality of output streams to the dissolution unit, the first output stream comprising a C12+ hydrocarbon.

Aspect 104. The plant according to Aspect 103, wherein the dissolution unit is further configured to: mix the feed comprising the waste plastic with the first output stream; and heat the feed comprising the waste plastic with the first output stream.

Aspect 105. The plant according to any one of Aspects 103 or 104, wherein the second separation unit is further configured to separate the depolymerization product effluent into: a second output stream comprising a C2-C4 hydrocarbon; and a third output stream comprising a C5-C11 hydrocarbon.

Aspect 106. The plant according to Aspect 105, further comprising: a C2-C4 olefin producing unit configured to receive the second output stream and to produce a C2-C4 olefin from the second output stream; a C5-C11 olefin producing unit configured to receive the third output stream and to produce a C5-C11 olefin from the third output stream; or a combination thereof.

In the foregoing, reference is made to aspects of the disclosure. However, it should be understood that the disclosure is not limited to specific described aspects. Instead, any combination of the following features and elements, whether related to different aspects or not, is contemplated to implement and practice the disclosure. Furthermore, although aspects of the disclosure may achieve advantages over other possible solutions and/or over the prior art, whether or not a particular advantage is achieved by a given aspect is not limiting of the disclosure. Thus, the foregoing aspects, features, embodiments, implementations, and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

As is apparent from the foregoing general description and the specific aspects, while forms of the aspects have been illustrated and described, various modifications may be made without departing from the spirit and scope of the present disclosure. Accordingly, it is not intended that the present disclosure be limited thereby. Likewise, the term “comprising” is considered synonymous with the term “including.” Likewise whenever a formulation, a composition, an element or a group of elements is preceded with the transitional phrase “comprising,” it is understood that we also contemplate the same formulation, composition or group of elements with transitional phrases “consisting essentially of,” “consisting of,” “selected from the group of consisting of,” or “Is” preceding the recitation of the formulation, composition, element, or elements and vice versa, for example, the terms “comprising,” “consisting essentially of,” “consisting of” also include the product of the combinations of elements listed after the term.

References cited herein are incorporated by reference herein in their entirety to indicate the state of the art as of their publication or filing date and it is intended that this information may be employed herein, if desired, to exclude specific aspects that are in the prior art.

For purposes of this present disclosure, and unless otherwise specified, all numerical values within the detailed description and the claims herein are modified by “about” or “approximately” the indicated value, and consider experimental error and variations that would be expected by a person having ordinary skill in the art. It will be further understood that there are a number of values disclosed therein, and that each value is also herein disclosed as “about” that particular value in addition to the value itself. In aspects, use of the term “about” may refer to ±20% of the stated value, ±15% of the stated value, ±10% of the stated value, ±5% of the stated value, ±3% of the stated value, ±2% of the stated value, or ±1% of the stated value.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited. For example, by disclosing a temperature of from 70° C. to 80° C., an intent is to recite individually 70° C., 71° C., 72° C., 73° C., 74° C., 75° C., 76° C., 77° C., 78° C., 79° C., and 80° C., including any sub-ranges and combinations of sub-ranges encompassed therein such that any of the foregoing numbers may be used singly to describe an open-ended range or in combination to describe a close-ended range. Moreover, all numerical end points of ranges disclosed herein are approximate, unless excluded by proviso. As a representative example, if one or more operations in the processes described herein may be conducted at a temperature in a range from 10° C. to 75° C., this range should be interpreted as encompassing temperatures in a range from “about” 10° C. to “about” 75° C.

A “composition” may include component(s) of the composition, reaction product(s) of two or more components of the composition, a remainder balance of remaining starting component(s), or combinations thereof.

As used herein, the indefinite article “a” or “an” shall mean “at least one” unless specified to the contrary or the context clearly indicates otherwise. For example, aspects comprising “a hydrocarbon” include aspects comprising one, two, or more hydrocarbons, unless specified to the contrary or the context clearly indicates only one hydrocarbon is included. For example, aspects comprising “a C12+ hydrocarbon” include aspects comprising one, two, or more C12+ hydrocarbons, unless specified to the contrary or the context clearly indicates only one hydrocarbon is included.

When a compound is described herein such that a particular isomer, enantiomer, or diastereomer of the compound is not specified, for example, in a formula or in a chemical name, that description is intended to include each isomer and enantiomer of the compound described individual or in any combination. For example, any general structure, formula, or name presented is also intended to encompass all structural isomers, conformational isomers, regioisomers, stereoisomers (such as enantiomers, diastereomers, and other optical isomers whether in enantiomeric or racemic forms, as well as mixtures of stereoisomers, as the context permits or requires) that may arise from a particular set of substituents, unless indicated otherwise. Thus, a general reference to a compound includes all structural isomers unless specified to the contrary or the context clearly indicates otherwise. For example, reference to a hydrocarbon without specifying a particular isomer (such as butyl) expressly discloses all isomers (such as n-butyl, iso-butyl, sec-butyl, and tert-butyl). For example, reference to a C5 hydrocarbon expressly discloses all isomers thereof.

The term “polymer” is used herein generically to include homopolymers, copolymers, terpolymers, and so forth, such as olefin homopolymers, copolymers, terpolymers, and the like. A copolymer may be derived from an olefin monomer and one olefin comonomer, while a terpolymer may be derived from an olefin monomer and two olefin comonomers. Accordingly, “polymer” encompasses copolymers, terpolymers, etc., derived from any olefin monomer and comonomer(s) disclosed herein. Similarly, an ethylene polymer would include ethylene homopolymers, ethylene copolymers, ethylene terpolymers, and the like. As an example, an olefin polymer (polyolefin), such as an ethylene copolymer, may be derived from ethylene and a comonomer, such as 1-butene, 1-hexene, or 1-octene. If the monomer and comonomer were ethylene and 1-hexene, respectively, the resulting polymer could be categorized an as ethylene/1-hexene copolymer or a poly(ethylene-co-1-hexene) polymer.

While the foregoing is directed to aspects of the present disclosure, other and further aspects of the disclosure may be devised without departing from the basic scope thereof, and the scope thereof is determined by the claims that follow.

What is claimed is:

1. A process for converting waste plastic, the process comprising:
heating, in a dissolution unit, a waste plastic feed with a dissolution medium to form a mixture comprising an insoluble component and a soluble component;
separating the soluble component of the mixture from the insoluble component of the mixture;
contacting the soluble component with a depolymerization catalyst under depolymerization conditions to form a depolymerization product effluent;
separating a C12+ hydrocarbon from the depolymerization product effluent; and
introducing the C12+ hydrocarbon to the dissolution unit to heat with the waste plastic feed.

2. The process according to claim 1, wherein, prior to the heating the waste plastic feed with the dissolution medium, the process further comprises:
removing at least a portion of water from the waste plastic feed;
removing at least a portion of O₂ from the waste plastic feed;
or a combination thereof.

3. The process according to claim 1, wherein the heating the waste plastic feed with the dissolution medium comprises selectively dissolving at least a portion of the waste plastic feed at a temperature that is about 250°C or less.

4. The process according to claim 1, wherein the waste plastic feed comprises a polyolefin.

5. The process according to claim 4, wherein the waste plastic feed further comprises a waste plastic other than the polyolefin in an amount that is about 10 wt % or less based on a total weight of the waste plastic feed.

6. The process according to claim 4, wherein the waste plastic other than the polyolefin comprises a polyester, a polyamide, a polyurethane, a polyphenol, a polycarbonate, a halogen-containing polymer, a polylactic acid, a polyacrylic acid, a polyacrylate, a polyacetal, or combinations thereof.

7. The process according to claim 1, wherein the waste plastic feed comprises a halogen content in an amount of about 36,000 ppm or less based on a total weight of the waste plastic feed.

8. The process according to claim 1, wherein:
the insoluble component comprises a waste plastic other than a polyolefin;
the insoluble component of the mixture comprises a rubber, a filler, a pigment, a dye, a process aid, a stabilizer, an ink, a plasticizer, a slip agent, an antiblock agent, an antioxidant, a UV stabilizer, a cellulose fiber, an adhesive, or combinations thereof; or
a combination thereof.

9. The process according to claim 1, wherein the dissolution medium comprises:
a hydrocarbon feed having a boiling point in a range from about 214° C. to about 680° C.;
a hydrocarbon feed comprising a petroleum-based material, a fossil fuel-based material, a bio-based material, or combinations thereof; or
a combination thereof.

10. The process according to claim 1, wherein the depolymerization catalyst comprises a zeolite-based catalyst, a chromia-based catalyst, a non-zeolite cracking catalyst, a solid acid catalyst, or combinations thereof.

11. The process according to claim 10, wherein:
when the depolymerization catalyst comprises the zeolite-based catalyst, the zeolite-based catalyst comprises L-zeolite (zeolite L or LTL), X-zeolite (zeolite X), Y-zeolite (Zeolite Y), omega zeolite, beta zeolite, SAPO-34 zeolite, USY Zeolite, HY zeolite, ZSM-4, ZSM-5 (MFI), ZSM-10, ZSM-11, ZSM-12, ZSM-20, ZSM-22, ZSM-23, ZSM-34, ZSM-35, ZSM-50, REY, USY, RE-USY, LZ-210, LZ-210-A, LZ-210-M, LZ-210-T, SSZ-13, SSZ-24, SSZ-26, SSZ-31, SSZ-33, SSZ-35, SSZ-37, SSZ-41, SSZ-42, SSZ-44, MCM-58, H-MOR (H-mordenite), mazzite, faujasite, chabazite, a modified mesoporous form thereof, an acid-modified form thereof, or combinations thereof;
when the depolymerization catalyst comprises the chromia-based catalyst, the chromia-based catalyst comprises an organic chromium compound, amorphous Cr₂O₃, crystalline Cr₂O₃, or combinations thereof;
when the depolymerization catalyst comprises the non-zeolite cracking catalyst, the non-zeolite cracking catalyst comprises clay, acid treated clay, perovskite, layered titanate, silica alumina, silica-coated alumina, acidic alumina, tungstated zirconia, activated carbon, natural kaolin, acid-modified kaolin, bentonite, or combinations thereof;
when the depolymerization catalyst comprises the solid acid catalyst, the solid acid catalyst comprises a solid oxide treated with an electron withdrawing anion; or
combinations thereof.

12. The process according to claim 1, wherein the depolymerization conditions comprise:
heating at a depolymerization temperature in a range from about 200° C. to about 300° C. while contacting the soluble component with the depolymerization catalyst;
contacting the soluble component with the depolymerization catalyst in the presence of hydrogen at a pressure in a range from about 689 kPa to about 2,069 kPa; or
a combination thereof.

13. The process according to claim 1, wherein the depolymerization product effluent comprises:
an amount of C2-C4 hydrocarbon in a range from about 65 wt % to about 98 wt % based on a total amount of C2-C4 hydrocarbon, C5-C11 hydrocarbon, C12+ hydrocarbon, and methane present in the depolymerization product effluent, the total amount of C2-C4 hydrocarbon, C5-C11 hydrocarbon, C12+ hydrocarbon, and methane present in the depolymerization product effluent equal to 100 wt %;
an amount of C5-C11 hydrocarbon in a range from about 1 wt % to about 30 wt % based on the total amount of C2-C4 hydrocarbon, C5-C11 hydrocarbon, C12+ hydrocarbon, and methane present in the depolymerization product effluent;
an amount of the C12+ hydrocarbon in a range from about 1 wt % to about 20 wt % based on the total amount of C2-C4 hydrocarbon, C5-C11 hydrocarbon, C12+ hydrocarbon, and methane present in the depolymerization product effluent; and
an amount of methane, if present, in a range from greater than 0 wt % to about 3 wt % based on the total amount of C2-C4 hydrocarbon, C5-C11 hydrocarbon, C12+ hydrocarbon, and methane present in the depolymerization product effluent.

14. The process according to claim 1, further comprising:
separating a C2-C4 hydrocarbon from the depolymerization product effluent; and
separating a C5-C11 hydrocarbon from the depolymerization product effluent.

15. The process according to claim 14, further comprising:
producing olefins from the C2-C4 hydrocarbon separated from the depolymerization product effluent;
producing olefins from the C5-C11 hydrocarbon separated from the depolymerization product effluent; or
a combination thereof.

16. A process for converting waste plastic to hydrocarbons, the process comprising:
heating, under dissolution conditions and in a dissolution unit, a waste plastic feed with a dissolution medium to form a dissolution product, the dissolution conditions comprising a temperature in a range from about 100° C. to about 200° C.;
performing a solid-liquid separation on the dissolution product to separate a soluble component of the dissolution product from an insoluble component of the dissolution product;
contacting, under depolymerization conditions, hydrocarbon polymers present in the soluble component with a depolymerization catalyst to form a depolymerization product effluent;
separating the depolymerization product effluent into at least three fractions comprising:
a first fraction comprising a C2-C4 hydrocarbon;
a second fraction comprising a C5-11 hydrocarbon; and
a third fraction comprising a C12+ hydrocarbon; and
combining the third fraction with the waste plastic feed in the dissolution unit.

17. A plant for converting waste plastic, the plant comprising:
a dissolution unit configured to heat a feed comprising waste plastic to provide a mixture comprising an insoluble component and a soluble component;
a first separation unit configured to receive the mixture from the dissolution unit and to separate the mixture;
a depolymerization reactor configured to receive the soluble component from the first separation unit and to contact the soluble component with a depolymerization catalyst under depolymerization conditions to form a depolymerization product effluent;
a second separation unit configured to receive the depolymerization product effluent from the depolymerization reactor and to separate the depolymerization product effluent into a plurality of output streams; and
a line configured to transfer a first output stream of the plurality of output streams to the dissolution unit, the first output stream comprising a C12+ hydrocarbon.

18. The plant according to claim 17, wherein the dissolution unit is further configured to:
mix the feed comprising the waste plastic with the first output stream; and
heat the feed comprising the waste plastic with the first output stream.

19. The plant according to claim 17, wherein the second separation unit is further configured to separate the depolymerization product effluent into:
a second output stream comprising a C2-C4 hydrocarbon; and
a third output stream comprising a C5-C11 hydrocarbon.

20. The plant according to claim 19, further comprising:
a C2-C4 olefin producing unit configured to receive the second output stream and to produce a C2-C4 olefin from the second output stream;
a C5-C11 olefin producing unit configured to receive the third output stream and to produce a C5-C11 olefin from the third output stream; or
a combination thereof.
